(19) United States
(12) Patent Application Publication
(10) Pub. No.: US 2017/0333421 A1
(43) Pub. Date: Nov. 23, 2017

(54) Population Pharmacokinetics of Liposomal Irinotecan

(71) Applicant: IPSEN BIOPHARM LTD., Wrexham (GB)

(72) Inventors: Bambang Adiwijaya, Belmont, MA (US); Jonathan Basil Fitzgerald, Arlington, MA (US)

(21) Appl. No.: 15/598,633

(22) Filed: May 18, 2017

Related U.S. Application Data
(60) Provisional application No. 62/478,295, filed on Mar. 29, 2017.
(60) Provisional application No. 62/450,800, filed on Jan. 26, 2017.
(60) Provisional application No. 62/433,687, filed on Dec. 13, 2016.
(60) Provisional application No. 62/338,324, filed on May 18, 2016.

(51) Int. Cl. A61K 31/4745; A61K 9/127; A61K 31/573; A61K 31/519; A61K 31/517; A61K 31/513; A61K 45/06; A61K 9/00

(52) U.S. Cl. CPC A61K 31/4745; A61K 31/517; A61K 9/1271; A61K 31/513; A61K 31/573; A61K 31/519; A61K 9/127; A61K 9/0019; A61K 45/06

(57) ABSTRACT

Nal-IRI is a liposomal formulation of irinotecan with a longer half-life (t_{1/2}), higher plasma total irinotecan (tIRI), and lower SN-38 maximum concentration (Cₘₐₓ) compared with non-liposomal irinotecan.

Population pharmacokinetic (PK) analysis of nal-IRI was performed for tIRI and total SN-38 (tSN38) using patient samples from 6 studies. PK-safety association was evaluated for neutropenia and diarrhea in 353 patients. PK-efficacy association was evaluated from a phase 3 study in pancreatic cancer NAPOLI1.

Efficacy was associated with longer duration of unencapsulated SN-38 (uSN38) above a threshold and higher C_{avg} of tIRI, tSN38 and uSN38. Neutropenia was associated with uSN38 Cₘₐₓ and diarrhea with tIRI Cₘₐₓ. Baseline predictive factors were race, BSA, and bilirubin.

Analysis identified PK factors associated with efficacy, safety, and predictive baseline factors. The results support the benefit of nal-IRI dose of 70 mg/m² (free base; equivalent to 80 mg/m² salt base) Q2W over 100 mg/m² Q3W.

### CROSS-REFERENCE TO RELATED APPLICATIONS

This U.S. Patent Application claims priority to U.S. Applications 62/338,324 filed on May 18, 2016, 62/433,687 filed on Dec. 13, 2016, 62/450,800 filed on Jan. 26, 2017, and 62/478,295 filed on Mar. 29, 2017, the disclosures of which are considered part of the disclosure of this application and are hereby incorporated by reference in their entirety.

### TECHNICAL FIELD

The present invention relates to the treatment of cancer with liposomal irinotecan.

### BACKGROUND

Nal-IRI is a liposomal formulation of irinotecan that is approved, in combination with 5-fluorouracil (5-FU) and leucovorin (LV), for the treatment of metastatic pancreatic cancer after progression following gemcitabine-based therapy. Nal-IRI has a longer half-life (t_{1/2}), higher plasma total irinotecan (tIRI), and lower SN-38 maximum concentration (Cₘₐₓ) compared with non-liposomal irinotecan.

Liposomal formulations have been investigated as a drug delivery system to modulate the pharmacological properties of small molecules. In cancer therapeutics, liposomal formulations can deposit in tumors through leaky vasculature by the enhanced permeability and retention effect (EPR), creating a local depot for drug release. Nanoliposomal irinotecan (nal-IRI, MM-398, PEP02, BAX2398) is a liposomal formulation of irinotecan for intravenous injection designed to combine the properties of long plasma circulation and increased delivery of irinotecan to tumor lesions via the EPR effect. The clinical benefit of nal-IRI was demonstrated in a Phase 3 study in patients with metastatic pancreatic cancer previously treated with a gemcitabine-based therapy (NAPOLI-1). Results showed that nal-IRI in combination with 5-fluorouracil (5-FU) and leucovorin (LV) significantly increased median overall survival (OS) compared with a 5-FU/LV control arm (6.1 and 4.2 months, respectively), with an unstratified hazard ratio (HR) of 0.67 (P=0.012). Additionally, the combination achieved a median progression-free survival (PFS) that approximately doubled that of the control arm (3.1 and 1.5 months, respectively; HR of 0.56; P=0.0001). As neutropenia and diarrhea are side effects that are associated with irinotecan, further investigation with nal-IRI is warranted.

The clinical pharmacokinetics of nal-IRI were previously compared with those of non-liposomal irinotecan (irinotecan HCl) in a Phase 2 study in patients with gastric cancer. Reanalysis of the data showed that compared with irinotecan HCl 300 mg/m² every 3 weeks [Q3W] (n=27), nal-IRI 100 mg/m² Q3W (n=37; free-base, equivalent to 120 mg/m² irinotecan hydrochloride trihydrate salt) had a total irinotecan (tIRI) maximum concentration (Cₘₐₓ) that was 13.4-times higher, a half-life (tin) that was 2.0 times longer, and an area under the concentration-time curve (AUC_{0-∞}) that was 46.2-times greater (Reanalysis by calculating geometric means instead of arithmetic means and by reporting the actual values instead of dose-normalized values). The t_{1/2} and AUC_{0 ∞}of SN-38, the active metabolite of irinotecan, were also increased relative to non-liposomal irinotecan (3.0- and 1.4-times, respectively), while maintaining a 5.3-times lower SN-38 Cₘₐₓ. In a separate clinical trial, nal-IRI-mediated tumor delivery were evaluated in tumor biopsies from 13 patients collected 72 h after the administration of 70 mg/m² nal-IRI. Total irinotecan (tIRI) in the tumor was 0.5-times those in the plasma, however, the total SN-38 (tSN38) were 6-times higher in tumor than in plasma, and the ratio of tSN38:tIRI (a measure of the extent of conversion) was 8-times higher in tumor than in plasma.

The extended plasma pharmacokinetics of liposomal formulations provides an opportunity to dissect the differences between derived pharmacokinetics parameters, including average concentration (C_{avg}) and Cₘₐₓ, and time above a threshold (t_{uSN38>thr}), and their association with efficacy and safety. With non-liposomal irinotecan, C_{avg} and Cₘₐₓ were highly correlated, and therefore, the dichotomization of the associations with efficacy and safety endpoints have been difficult to elucidate.

### SUMMARY

The dichotomization of the associations between C_{avg} or Cₘₐₓ with efficacy and safety endpoints for liposomal irinotecan has successfully been determined. Analysis of the data from all patients treated with nal-IRI was performed to better understand the association of the prolonged pharmacokinetics of nal-IRI with efficacy (OS and PFS; NAPOLI-1) endpoints and on the incidence and severity of the most common adverse events (AEs). Baseline factors predictive of plasma pharmacokinetics were also determined.

Liposomal encapsulation of irinotecan (nal-IRI) extends the half-lives of irinotecan and SN-38, the active metabolite of irinotecan. Through population pharmacokinetic analysis and modeling, it was shown that efficacy was associated with the average concentration of SN-38 and the duration of time SN-38 was above a certain threshold, while safety was associated with maximum concentrations. These results support the choice of a 70 mg/m² every-2-weeks nal-IRI dose for patients with metastatic pancreatic cancer previously treated with gemcitabine-based therapy to improve safety while maintaining efficacy compared with a dose regimen of 100 mg/m² every 3 weeks.

In one aspect, the invention includes a method of treating cancer in a human patient, the method comprising administering to the human patient in need thereof irinotecan liposome in a dose and dose interval that are both therapeutically tolerable and therapeutically effective, wherein
a. the therapeutically tolerable dose and dose interval is selected based on the maximum SN38 plasma concentration and the maximum total irinotecan concentration in the plasma of the patient (in some embodiments, the maximum SN38 plasma concentration is within a range selected from column A of Table A, and the maximum total irinotecan concentration in the plasma of the patient is within a range selected from column B of Table A), and
b. the therapeutically effective dose and dose interval is selected based on the time of SN38 plasma concentration above a cancer indication-specific threshold concentration, and the average SN38 plasma concentration of the patient (in some embodiments, the time of SN38 plasma concentration above a cancer indication-specific threshold concentration is within a range selected from column C of Table A, and the average SN38 plasma concentration of the patient is within a range selected from column D of Table A).

In one embodiment of this aspect, the irinotecan liposome is MM-398.

In one embodiment, the therapeutically tolerable dose and dose interval are selected to provide a minimal predicted incidence of neutropenia and diarrhea at a given therapeutically effective dose and dose interval.

In some embodiments, the cancer comprises a solid tumor in the human patient. In a further embodiment, the cancer is pancreatic cancer.

In another aspect, the invention includes a method of treating cancer in a human patient, the method comprising administering to the human patient in need thereof irinotecan liposome in a first dose that is both therapeutically tolerable and therapeutically effective, followed by administering a second dose of the irinotecan liposome at a first dose interval after the first dose, wherein the second dose and first dose interval are selected based on:
a. the maximum unencapsulated SN38 plasma concentration and the maximum total irinotecan concentration in the plasma of the patient (in some embodiments, the maximum SN38 plasma concentration is within a range selected from column A of Table A, and the maximum total irinotecan concentration in the plasma of the patient is within a range selected from column B of Table A), and
b. the therapeutically effective dose and dose interval is selected based on the time of SN38 plasma concentration above a cancer indication-specific threshold concentration, and the average SN38 plasma concentration of the patient (in some embodiments, the time of SN38 plasma concentration above a cancer indication-specific threshold concentration is within a range selected from column C of Table A, and the average SN38 plasma concentration of the patient is within a range selected from column D of Table A).

In another embodiment, the method further comprises measuring the total irinotecan and the SN-38 in the plasma of the human patient after the first dose and before the second dose.

In another embodiment, the method further comprises administering a third dose following a second dose interval after the second dose, wherein the second dose interval is determined using the measurement of the total irinotecan and the SN-38 in the plasma of the human patient after the first dose and before the second dose (in some embodiments, the maximum SN38 plasma concentration is within a range selected from column A of Table A, and the maximum total irinotecan concentration in the plasma of the patient is within a range selected from column B of Table A).

In one embodiment,
a. the maximum SN38 plasma concentration is from 0.1 ng/mL to 25 ng/mL (for example, from 0.4 ng/mL to 15 ng/mL, or from 0.6 ng/mL to 10 ng/mL, or from 0.8 ng/mL to 7 ng/mL, or from 0.88 ng/mL to 5.98 ng/mL, or from 0.8 ng/mL to 2.0 ng/mL, or from 2.0 ng/mL to 3.5 ng/mL, or from 3.5 ng/mL to 5.0 ng/mL, or from 5.0 ng/mL to 6.5 ng/mL),
b. the maximum total irinotecan concentration in the plasma of the patient is from 5 mg/L to 200 mg/L (for example from 10 mg/L to 100 mg/L, or from 15 mg/L to 70 mg/L, or from 18.0 mg/L to 60 mg/L, or from 18.9 mg/L to 53.1 mg/L, or from 18.0 mg/L to 25 mg/L, or from 25 mg/L to 35 mg/L, or from 35 mg/L to 45 mg/L, or from 45 mg/L to 55 mg/L),
c. the time of SN38 plasma concentration above the threshold concentration of, for example, 0.01 ng/mL, 0.02 ng/mL, 0.03 ng/mL, 0.04 ng/mL, 0.05 ng/mL, 0.06 ng/mL, 0.07 ng/mL, 0.08 ng/mL, 0.09 ng/mL, 0.10 ng/mL, 0.11 ng/mL, 0.12 ng/mL, 0.13 ng/mL, 0.14 ng/mL, or 0.15 ng/mL in the first 6 weeks is from 1 to 6 weeks (for example from 1.5 to 6 weeks, or from 1.94 to 6.00 weeks), and
d. the average SN38 plasma concentration of the patient is from 0.1 ng/mL to 20 ng/mL (for example, from 0.15 ng/mL to 10 ng/mL, or from 0.18 ng/mL to 4 ng/mL, or from 0.20 to 1.66 ng/mL, or from 0.18 ng/mL to 0.50 ng/mL, or from 0.0.50 ng/mL to 0.80 ng/mL, or from 0.80 ng/mL to 1.10 ng/mL, or from 1.10 ng/mL to 1.40 ng/mL, or from 1.40 ng/mL to 1.70 ng/mL).

TABLE A

| | | Column C: | |
|---|---|---|---|
| | | Ranges of time | |
| Column A: | Column B: | of SN38 plasma | Column D: |
| Ranges of | Ranges of | concentration above | Ranges of |
| maximum | maximum total | the threshold | average |
| SN38 plasma | irinotecan | concentration in the | SN38 plasma |
| concentration | concentration | first 6 weeks | concentration |
| (ng/mL) | (mg/L) | (weeks) | (ng/mL) |
| 0.1-25 | 5-200 | 1-6 | 0.1-20 |
| 0.4-15 | 10-100 | 1.5-6 | 0.15-10 |
| 0.6-10 | 15-70 | 1.94-6 | 0.18-4 |
| 0.8-7 | 18.0-60 | | 0.20-1.66 |
| 0.88-5.98 | 18.9-53.1 | | 0.18-0.50 |
| 0.8-2.0 | 18.0-25 | | 0.50-0.80 |
| 2.0-3.5 | 25-35 | | 0.80-1.10 |
| 3.5-5.0 | 35-45 | | 1.10-1.40 |
| 5.0-6.5 | 45-55 | | 1.40-1.70 |

### BRIEF DESCRIPTION OF THE FIGURES

FIG. 1 is a diagram of nal-IRI PK model.

FIG. 2 is a line graph showing the mean concentrations of total irinotecan, SN-38 and SN-38G over one week after the administration of either nal-IRI (100 mg/m² based on irinotecan as free base, equivalent to 120 mg/m² based on irinotecan as the hydrochloride trihydrate) or non-liposomal irinotecan (300 mg/m²) in Study PEP0206.

FIG. 3 provides the observed and predicted typical plasma concentration profile of total irinotecan and SN-38 in patients administered nal-IRI 70 mg/m² Q2W or nal-IRI 100 mg/m² Q3 W.

FIG. 4A is a series of graphs providing observed and model-fitted concentrations by time and by dose for tIRI on a logarithmic scale.

FIG. 4B is a series of graphs providing observed and model-fitted concentrations by time and by dose for SN-38 on a logarithmic scale.

FIG. 4C is a series of graphs providing observed and model-fitted concentrations by time and by dose for tIRI on a logarithmic scale.

FIG. 4D is a series of graphs providing observed and model-fitted concentrations by time and by dose for SN-38 on a logarithmic scale.

FIG. 5 is a Kaplan-Meier Plot of overall survival by quartiles of un-encapsulated SN-38 (uSN38) time above threshold in the nal-IRI+5-FU/LV arm of NAPOLI-1.

FIG. 6 is a Kaplan-Meier Plot of overall survival by quartiles of unencapsulated SN-38 (uSN38) time above threshold in the nal-IRI monotherapy arm of NAPOLI-1.

FIG. 7 is a plot showing the association between best response and time (uSN38>thr) for nal-IRI+5-FU/LV arm in NAPOLI-1.

FIG. 8 is a Kaplan-Meier Plot of overall survival by quartiles of unencapsulated SN-38 (uSN38) average concentration in the nal-IRI+5FULV arm of NAPOLI-1.

FIG. 9A is a plot showing the incidence rates of neutropenia of a grade≧3 by plasma pharmacokinetics in patients treated with nal-IRI.

FIG. 9B is a plot showing the incidence rates of diarrhea of a grade≧3 by plasma pharmacokinetics in patients treated with nal-IRI.

FIG. 10 is a forest plot of total irinotecan maximum concentration (Cmax) by baseline covariate subgroups.

FIG. 11 is a forest plot of un-encapsulated SN-38 maximum concentration (Cmax) by baseline covariate subgroups.

FIG. 12 is a graphic providing selected baseline factors and associated plasma total irinotecan and un-encapsulated SN-38 Cmax with nal-IRI 70 mg/m².

FIG. 13 is a set of graphs of PK of MM-398 in Tumor Biopsies.

FIG. 14 is a set of graphs showing MM-398 extends SN-38 exposure duration in tumors, which is predictive of activity.

FIG. 15 provides a chart of the hybrid tumor PK model supported 80 mg/m² (salt) q2w.

FIG. 16 is a model to simulate SN-38 PK.

FIG. 17 is a plot of Overall Survival by unencapsulated SN38 C_{avg} Quartiles.

FIG. 18A is a multiplot linear graph of an in vitro cell line response model to a varying concentration of SN-38.

FIG. 18B is a multiplot non-linear graph of an in vitro cell line response model to a varying concentration of SN-38.

FIG. 18C is a bar graph showing the relative error for both the linear and non-linear graphs, wherein the leftmost bar is the linear error and the rightmost bar is the non-linear error for both cell lines (CFPAC1 and BXPC3).

FIG. 19 is a plot providing the tumor concentration of SN-38 and CPT-11 during several CPT-11 dosing cycles in a xenograft tumor growth response model, which combines the pharmacokinetic and in vitro cell line models.

FIG. 20A is a plot showing the overall volume change of an untreated tumor (control) over days in a xenograft tumor growth response model, which combines the pharmacokinetic and in vitro cell line models.

FIG. 20B is a plot showing the overall volume change of a tumor treated with nal-IRI over days in a xenograft tumor growth response model, which combines the pharmacokinetic and in vitro cell line models.

### DETAILED DESCRIPTION

Glossary of Abbreviations

| Abbreviation | Terms |
|---|---|
| 5-FU | 5-fluorouracil |
| LV | leucovorin |
| nal-IRI | nanoliposomal irinotecan |
| tIRI | total irinotecan |
| eSN38 | encapsulated SN-38 |
| tSN38 | total SN-38 |
| uSN38 | unencapsulated SN-38 |
| t_{uSN38>thr} | time when SN-38 is above a threshold concentration |
| OS | overall survival |
| PFS | progression-free survival |
| PK | pharmacokinetics |

Definitions

As used herein, the term “MM-398”, which has the tradename “Onivyde™” is a liposomally encapsulated form of irinotecan. Irinotecan has the chemical name “(S)-4,11-diethyl-3,4,12,14-tetrahydro-4-hydroxy-3,14-dioxo1H-pyrano[3′,4′:6,7] indolizino[1,2-b]quinolin-9-yl-[1,4′bipiperidine]-1′-carboxylate” and the following chemical formula:

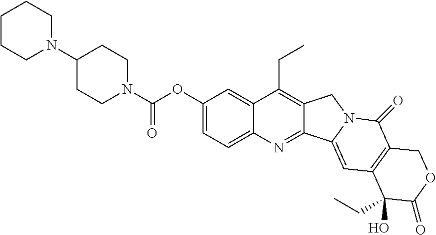 [SMILES: CCc1c2c(nc3ccc(OC(=O)N4CCC(N5CCCCC5)CC4)cc13)-c1cc3c(c(=O)n1C2)COC(=O)[C@]3(O)CC]

The MM-398 liposome is a unilamellar lipid bilayer vesicle, approximately 110 nm in diameter, which encapsulates an aqueous space containing irinotecan in a gelated or precipitated state as the sucrose octasulfate salt. The vesicle is composed of 1,2-distearoyl-sn-glycero-3-phosphocholine (DSPC), cholesterol, and methoxy-terminated polyethylene glycol (MW 2000)-distearoylphosphatidyl ethanolamine (MPEG-2000-DSPE) in a 3:2:0.015 molar ratio, respectively.

In some embodiments, MM-398 is administered as an aqueous composition comprising MM-398, HEPES buffer, and sodium chloride. In some embodiments, 10 mL of the aqueous composition contains 43 mg of irinotecan. In some embodiments, the composition comprises irinotecan 4.3 mg/mL, 1,2-distearoyl-sn-glycero-3-phosphocholine (DSPC) 6.81 mg/mL, cholesterol 2.22 mg/mL, and methoxy-terminated polyethylene glycol (MW 2000)-distearoylphosphatidyl ethanolamine (MPEG-2000-DSPE) 0.12 mg/mL. In a further embodiment, the composition further comprises HEPES buffer 4.05 mg/mL and sodium chloride 8.42 mg/mL.

As used herein, the drug “5-fluorouracil” is an anticancer drug sold under various trade names, such as Adrucil, Carac, Efudex, and Efudix. 5-fluorouracil has the chemical structure:

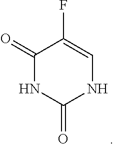

As used herein, the drug “leucovorin” is used in combination with 5-fluorouracil to treat cancer. Leucovorin has the structure:

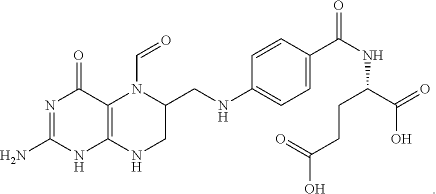 [SMILES: Nc1nc(=O)c2c([nH]1)NCC(CNc1ccc(C(=O)N[C@@H](CCC(=O)O)C(=O)O)cc1)N2C=O]

As used herein, the term “total irinotecan” refers to the total amount of irinotecan in a patient, whether the irinotecan is encapsulated in a liposome, or unencapsulated.

As used herein, the term “SN-38” is a biologically active antineoplastic drug, and is the active metabolite of irinotecan. SN-38 has the structure:

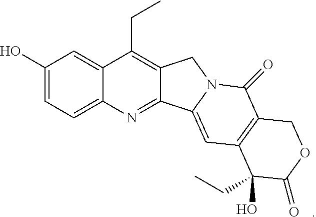 [SMILES: CCc1c2c(nc3ccc(O)cc13)-c1cc3c(c(=O)n1C2)COC(=O)[C@]3(O)CC]

As used herein, the term “total SN-38” refers to the total amount of SN-38 in a patient, whether the SN-38 is encapsulated in a liposome, or unencapsulated.

As used herein, the term “overall survival” is defined as the time from the date of patient randomization to date of death or the date last known alive.

As used herein, the term “progression-free survival” is defined as the number of months from the date of randomization to the date of death or progression, whichever occurred earlier.

MM-398 is a nanoliposomal irinotecan (nal-IRI). This study characterized the population PK and exposure-response with MM-398 in patients with solid tumors.

Methods:

Population pharmacokinetic analysis of nal-IRI was performed for tIRI and total SN-38 (tSN38) using patient samples from 6 clinical studies. Unencapsulated SN-38 (uSN38) was predicted from a model. Pharmacokinetic-safety association was evaluated for neutropenia and diarrhea in a pooled dataset (N=353). Pharmacokinetic-efficacy association was evaluated for OS, progression-free survival (PFS) and objective response rate using data from a phase 3 study in pancreatic cancer.

Patients and Methods

Patients and Treatment

Data were prospectively collected from patients enrolled in 6 trials that evaluated the effect of nal-IRI on a variety of tumor types, including colorectal, gastric, and pancreatic cancers (Table 1). Detailed eligibilities, methods and clinical results of these studies have been described previously. For example, the eligibility criteria in study NAPOLI-1 included adequate bone marrow reserve (absolute neutrophil count [ANC]>1500 cells/μL, platelet count>10⁶ cells/μL, hemoglobin>9g/dL), adequate renal function (serum creatinine [SCr]≦1.5 upper limit of normal [ULN]), and adequate liver function (bilirubin≦ULN, albumin≧3.0 g/dL; aspartate aminotransferase [AST] and alanine aminotransferase [ALT] of ≦2.5 ULN or ≦5 ULN if liver metastases were present). The nal-IRI doses in these studies were calculated based on the equivalent dose of irinotecan hydrochloride trihydrate; the doses described are based on irinotecan as free base (i.e., 70 mg/m² of irinotecan as the free base is equivalent to 80 mg/m² of irinotecan as the hydrochloride trihydrate). The final population pharmacokinetic dataset consisted of 353 subjects. Two subjects from NAPOLI-1 with tIRI but without tSN38 measurements were excluded from the analyses (Table 2).

TABLE 1
Clinical Pharmacology Studies in the Population Pharmacokinetic
Analysis

| | | | nal-IRI | | | |
|---|---|---|---|---|---|---|
| Study | | | Dose and | | Pharmacokinetic | |
| Number | | | Regimen, | Drugs in | Sample | |
| (Reference) | Indication | N | mg/m^{2a} | Combination | Collections | Analytes |
| PEP0201 | Solid | 11 | 50, 100 | None | Cycle 1: 0 | tIRI, |
| | tumors | | or 160 | Monotherapy | (predose), 0.5, | encapsulated |
| | | | (60, 120 | | 1.0, 1.5, 2.5, 3.5, | irinotecan, |
| | | | or 180) | | 4.5, 7.5, 10.5, | tSN38 |
| | | | q3w | | 13.5, 25.5, 49.5, | |
| | | | | | 73.5 and 169.5 hr | |
| | | | | | post drug infusion | |
| | | | | | Cycle 2: 0 | |
| | | | | | (predose) | |
| PEP0203 | Solid | 16 | 50, 70 | 5-FU/LV | Cycle 1: 0 | tIRI and |
| | tumors | | 90, 100 | | (predose), 0.5, | tSN38 |
| | | | (60, 80 | | 1.0, 1.5, 2.5, 4.5, | |
| | | | 100, 120) | | 10.5, 25.5, 49.5, | |
| | | | q3w | | 73.5 and 169.5 hr | |
| | | | | | post drug infusion | |
| | | | | | Cycle 2: 0 (pre- | |
| | | | | | dose) | |
| PEP0206 | Gastric | 37 | 100 | None | Cycle 1: 0 | tIRI, tSN38 |
| | and GEJ | | (120) | (monotherapy) | (predose), 0.5, | SN38G |
| | | | q3w | | 1.0, 1.5, 2.5, 4.5, | |
| | | | | | 10.5, 25.5, 49.5, | |
| | | | | | 73.5 and 169.5 hr | |
| | | | | | post drug infusion | |
| | | | | | Cycle 2: 0 (pre- | |
| | | | | | dose) | |
| PIST-CRC- | Colorectal | 18 | 60, 80, | None | Cycle 1: 0 | tIRI, tSN38 |
| 01 | | | 90 | (monotherapy) | (predose), 0.5, | |
| | | | (80, 90, | | 1.0, 1.5, 2.5, 4.5, | |
| | | | 100) q2w | | 10.5, 25.5, 49.5, | |
| | | | | | 73.5, 169.5 hr | |
| | | | | | post drug infusion | |
| NAPOLI-1 | Metastatic | 260 | Arm 2: | Arm 2: None | Cycle 1:0 | tIRI, tSN38, |
| | pancreatic | | 100 (120) | (monotherapy) | (predose), 1.5, | SN-38G, 5- |
| | cancer | | q3w | Arm 3: 5- | 2.5, 48 (Arm 3 | FU |
| | | | Arm 3: | FU/LV | only) and 168 hr | |
| | | | 70 (80) | | | |
| | | | q2w | | | |
| CITS | Solid | 13 | 70 (80) | None | Cycle 1: 0 | tIRI, tSN38 |
| (nal-IRI-01- | tumors | | q2w | (monotherapy) | (predose), 1.5, 3, | and SN-38G |
| 01-02) | | | | | 72 and 168 hr | |
| | | | | | Cycle 2: 0 | |
| | | | | | (predose) | |

^{a} Dose is given based on irinotecan free base. The original protocol dose based on irinotecan hydrochloride trihydrate, is in parentheses.
FU = fluorouracil;
q2w = administered every 2 weeks;
q3w = administered every 3 weeks;
SN-38G = glucuronidated SN-38;
tIRI = total irinotecan.

TABLE 2
Number of concentration samples in the data set

| | Number of | | | |
|---|---|---|---|---|
| | Subjects with | | | |
| | Collections for | Number of | Number of | Number of |
| | Pharmacoki- | Subjects | IRI | SN-38 |
| Study (Reference) | netic Analysis | Treated | Samples | Samples |
| Total before | 355 (IRI) | | 1808 | 1789 |
| removal of | 353 (SN-38) | | | |
| outliers | | | | |
| Total after | 355 (IRI) | | 1792 | 1765 |
| removal of | 353 (SN-38) | | | |
| outliers | | | | |
| CITS | 13 | 13 | 66 | 66 |
| (nal-IRI-01-01-02) | | | | |
| NAPOLI-1 | 260 (IRI) | 266 | 847 | 841 |
| | 258 (SN-38) | | | |
| PEP0201 | 11 | 11 | 120 | 117 |
| PEP0203 | 16 | 16 | 167 | 155 |
| PEP0206 | 37 | 44 | 393 | 388 |
| PIST-CRC-01 | 18 | 18 | 199 | 198 |

IRI = Irinotecan

Pharmacokinetic Data

Pharmacokinetic sample collection consisted of intense sampling during the first cycle of study drug administration in early studies and sparse sampling in the Phase 3 study NAPOLI-1 (Table 1). The analytes measured include tIRI (encapsulated plus unencapsulated irinotecan) and its active metabolite SN-38. In the first study, the levels of encapsulated irinotecan were found to be indistinguishable from total irinotecan; therefore, only total irinotecan levels were measured in the subsequent studies.

Covariate analysis was conducted using full covariate approach. Baseline patient information evaluated to predict plasma pharmacokinetics included body size (body surface area [BSA]), demographics, hepatic and renal function, pharmacogenomics, and extrinsic factors such as product manufacturing site and coadministration with 5-FU. Laboratory measurements (ALT, AST, bilirubin, SCr, and albumin) were log-transformed (log-normal distributions were observed) (Table 3). Liver metastasis status was only available from NAPOLI-1; therefore, the values for the other studies were imputed to be equal to “No”, and the effect of this imputation was evaluated in a sensitivity analysis. The estimated clearance of IRI was added as a covariate to the SN-38 input flux. Mechanistically, increased clearance of IRI was hypothesized to generate more release of unencapsulated irinotecan that would be available for in vivo conversion to SN-38 (clearance of nal-IRI likely results in broken liposome and release of irinotecan).

TABLE 3
Covariate Structure of the population pharmacokinetic analysis

| Analyte | Covariates | Parameters | Rationale |
|---|---|---|---|
| Total | Manufacturing site | Volume | Potential difference in |
| irinotecan | | | manufacturing of nal-IRI |
| | | | in phase 2 and phase 3 |
| | | | studies |
| | BSA | Volume | Body size, nal-IRI was |
| | | | dosed per BSA |
| | Age, sex, race | Clearance | Standard covariates |
| | Manufacturing site | Clearance | Potential difference in |
| | | | manufacturing of nal-IRI |
| | | | in phase 2 and phase 3 |
| | | | studies |
| | Coadministration | Clearance | Effect of |
| | with 5-FU | | co-administration |
| | | | (potential drug |
| | | | interactions) |
| | AST, ALT, | Clearance | Hepatic function |
| | bilirubin, albumin, | | |
| | liver metastasis | | |
| | Creatinine clearance | Clearance | Renal function |
| SN-38 | Manufacturing site | Fraction | Potential difference in |
| | | eSN38 in | manufacturing of nal-IRI |
| | | tIRI (f_{SN38}) | in phase 2 and phase 3 |
| | | | studies |
| | BSA | Conversion | Body size, nal-IRI was |
| | | rate (Kcov) | dosed per BSA |
| | Manufacturing site | Conversion | Potential difference in |
| | | rate (Kcov) | manufacturing of nal-IRI |
| | | | in Phase 1 and 2 versus |
| | | | Phase 3 studies |
| | IRI clearance | Conversion | Clearance of IRI is a |
| | | rate (Kcov) | surrogate measure of the |
| | | | activity of mononuclear |
| | | | phagocyte system (MPS), |
| | | | which may also affect the |
| | | | metabolism and |
| | | | formation rate of SN-38 |
| | Age, sex, race | Clearance | Standard covariates |
| | Coadministration | Clearance | Effect of |
| | with 5-FU | | coadministration |
| | AST, ALT, | Clearance | Hepatic function |
| | bilirubin, albumin, | | |
| | liver metastasis, | | |
| | Creatinine clearance | Clearance | Renal function |
| | UGT1A1*28 | Clearance | Glucuronidation step |
| | polymorphism | | (historical factor for |
| | | | nonliposomal irinotecan) |

ALT = alanine aminotransferase; AST = aspartate aminotransferase; BSA = body surface area; IRI = irinotecan; FU = fluorouracil.

Population Pharmacokinetic Modeling Analysis Methods

Modeling Assumptions

The nonlinear mixed effect modeling (NONMEM) was used to analyze the pharmacokinetic data of tIRI and tSN38 in patients administered nal-IRI. To account for measured values below the detection limit, the M3 method was implemented with concentrations in log-transformed values using the Laplacian estimation method.

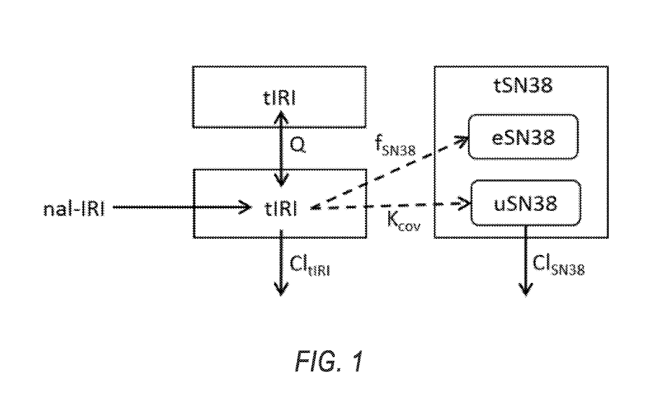

A diagram of the PK models of tIRI and tSN38 was shown in FIG. 1. In the figure, the PK model for nal-IRI consists of two components:1) tIRI and 2) tSN38. The tIRI model is a two-compartment model with first order elimination (Cl_{tIRI}). The tSN38 model consists of a sum of eSN38 and uSN38, with eSN38 as a constant (time-invariant) fraction of tIRI, and uSN38 is formed from a first-order conversion of tIRI (which represents two major steps into one process: a release of irinotecan from liposomal encapsulation, and a conversion of free irinotecan to SN-38). These two models are sequential models: tIRI model is independent, and tSN38 model depends on the results of the tIRI model. The independency of tIRI is because of the observed four orders of magnitude difference in plasma concentrations of tIRI and tSN38. In FIG. 1, tIRI is total irinotecan, tSN38 is total SN-38, eSN38 is encapsulated SN-38, uSN38 is unencapsulated SN-38, Cl_{tIRI} is plasma clearance rate for tIRI, Q is inter-compartmental clearance rate for tIRI, fsN₃₈ is the fraction of eSN-38 in ng per tIRI in μg, K_{cov} is the conversion of tIRI to uSN38, Cl_{SN38} is the plasma clearance rate for uSN38.

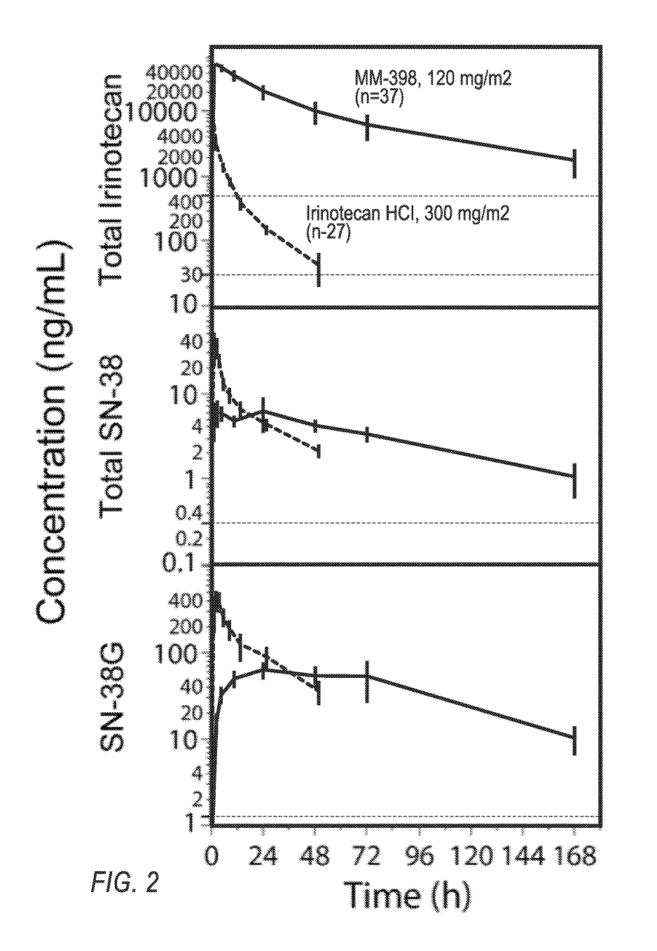

The final model of tIRI was a two-compartment model with first order elimination, and the tSN38 depends on tIRI model. tSN38 was represented as a sum of unencapsulated SN-38 (uSN38) and encapsulated SN-38 (eSN38), with eSN38 as a time-invariant fraction of tIRI, and uSN38 as a one-compartment model with first order production rate representing the process of release of irinotecan and its conversion to SN-38. The existence of eSN38 was supported by in vitro measurements and by the observation of delayed metabolism of SN-38 with nal-IRI administration. In study PEP0206, delayed appearance of SN-38G relative to the appearance of SN-38 was observed after nal-IRI administration, in contrast to the immediate appearance of SN-38G and SN-38 after non-liposomal irinotecan administration (FIG. 2). In FIG. 2, Error bars indicate 95% confidence interval. Dotted lines indicate lower limit of quantification (LLOQ); total irinotecan measurements consist of two LLOQ values because two different irinotecan assays were used to measure low and high range of concentrations. The concentrations less than LLOQ values were set to the corresponding LLOQ.

This observation supports the hypothesis that only the uSN38 is bioavailable for glucuronidation. The fraction of eSN38 in tIRI was estimated to be 0.01%, which is comparable to the in-vitro measurement of 0.015% and is below the specification limit of irinotecan manufacturing. The inclusion of the uSN38 and eSN38 improved the model fitting (Table 4).

TABLE 4
Evolution of Pharmacokinetic Models in Development
and their Corresponding Objective Functions

| | | | Objective |
|---|---|---|---|
| Analyte | Run | Model Descriptions | functions |
| tIRI | 1 | Base model: two compartmental model | −2052.35 |
| | | without covariates | |
| | 2 | Final model: two compartmental model | −2183.76 |
| | | with full covariates | |
| | 3 | Comparator model: One compartment model | −1973.68 |
| | | (ADVAN1) without covariates | |
| | 4 | Comparator model: model 2 with an | −2185.40 |
| | | additional BSA-CL relationship | |
| tSN38 | 1 | Base model: one compartmental model with | −2687.32 |
| | | encapsulated and unencapsulated forms of | |
| | | SN-38 without covariates | |
| | 2 | Final model: one compartment model with | −3117.05 |
| | | encapsulated and unencapsulated forms of | |
| | | SN-38 with full covariates | |
| | 3 | Comparator model 1: model 2 without | −495.02 |
| | | considering SN-38 encapsulated and | |
| | | unencapsulated forms | |

tIRI = total irinotecan.
tSN38 = total SN-38

Simulation Analysis Methods

Simulations from post-hoc parameter estimates were used to derive pharmacokinetic parameters for the first cycle of nal-IRI, including the C_{avg} and Cₘₐₓ for tIRI, tSN38, and uSN38, as well as the time when uSN38 concentrations were greater than a threshold of 0.03 ng/mL in the first 6 weeks, which was measured since nal-IRI activity is strongly associated with the duration of exposure of SN-38 above a minimum inhibitory concentration. The threshold of 0.03 ng/mL was chosen based on the median IC₅₀ of SN-38 in in-vitro pancreatic cell lines (different choices of threshold of 0.02 to 0.3 ng/mL resulted in similar OS concordance indices). A sensitivity analysis was conducted to account for the contribution of dose modifications by multiplying the first-cycle C_{avg} with the fraction of total planned dose for the NAPOLI-1 study. For the evaluation of association between baseline covariate and pharmacokinetics, predicted concentrations were used based on a simulated dose of 70 mg/m². For the evaluation of fixed- and BSA-based dosing, predicted concentrations were based on the simulated dose of 70 mg/m² every 2 weeks, or 116.7 mg every 2 weeks (equivalent dose for a subject with median BSA).

One subject had very low tIRI concentrations (i.e., predicted C_{avg} of 10⁻⁶ mg/L; these values were artifacts of numerical precisions in the simulation). To reduce the potential that this outlier might affect the slope in the regression analysis, the IRI concentrations for this subject were set at 1-log₁₀ of the lower limit of quantification.

Exposure-Efficacy Analysis Methods

Pharmacokinetics-efficacy analysis was performed for each treatment arm (Arm 2 and Arm 3; Table 1) of NAPOLI-1. The associations between pharmacokinetic parameters and survival endpoints were measured using the concordance index, a metric to assess the degree of fit in a survival analysis. The selection of pharmacokinetic parameters was based on the magnitude of the concordance index and the positive direction of the association.

Exposure-Safety Analysis Methods

The safety dataset included patients from all 6 clinical studies (Table 1) and was evaluated for diarrhea and neutropenia, the most common AEs of interest in patients who receive irinotecan. To ensure an established, systematic clustering of AE terms reported, specialized grouping based on individual MedDRA version 14.1 terms was used for diarrhea and neutropenia (Table 5). The reported AEs included any grade and grade≧3 according to the NCI Common Terminology Criteria for Adverse Events 4.0. Two types of safety endpoints were evaluated: A) the incidence of treatment-emergent adverse events (TEAEs) implemented as the probability of occurrence (logistic regression); and B) the time to the first occurrence of TEAE implemented as a survival analysis. Because the conclusions were similar for both the incidence of AEs and the time to first occurrence of AEs, only the associations to the incidence of AEs are reported. The occurrence of repeated AEs within a subject was small (2% for diarrhea grade≧3 and 4% for neutropenia grade≧3), thus only the first occurrence of AEs was used, and repeated time-to-event analysis was not conducted.

TABLE 5
Definition of Clustering of Adverse Event Terms of Special Interest

| Adverse Event of | | |
|---|---|---|
| Special Interest | Individual MedDRA Preferred Terms in the Grouping | |
| Neutropenia | Agranulocytosis | Myelocyte count decreased |
| (product specific, | Band neutrophil count decreased | Myelocyte percentage decreased |
| subgroup of | Band neutrophil percentage | Myeloid maturation arrest |
| Myelosuppression | decreased | |
| MedDRA SMQ) | Cyclic neutropenia | Neutropenia |
| | Febrile neutropenia | Neutropenia neonatal |
| | Full blood count abnormal | Neutropenic infection |
| | Granulocyte count decreased | Neutropenic sepsis |
| | Granulocytes maturation arrest | Neutrophil count abnormal |
| | Granulocytopenia | Neutrophil count decreased |
| | Idiopathic neutropenia | Neutrophil percentage decreased |
| | Metamyelocyte count decreased | Pancytopenia |
| | Myeloblast count decreased | Promyelocyte count decreased |
| | Myeloblast percentage decreased | |
| Diarrhea | Fecal containment device | Abnormal feces |
| (Noninfectious | insertion | |
| diarrhea MedDRA | Fecal incontinence | Antidiarrheal supportive care |
| SMQ) | Fecal volume increased | Bowel movement irregularity |
| | Feces discolored | Change of bowel habit |
| | Frequent bowel movements | Colitis |
| | Gastroenteritis | Colitis erosive |
| | Gastroenteritis eosinophilic | Colitis ischemic |
| | Gastroenteritis radiation | Colitis microscopic |
| | Gastrointestinal hypermotility | Colitis psychogenic |
| | Gastrointestinal inflammation | Culture stool negative |
| | Gastrointestinal motility disorder | Defecation urgency |
| | Gastrointestinal toxicity | Diarrhea |
| | Gastrointestinal tract irritation | Diarrhea hemorrhagic |
| | Intestinal transit time abnormal | Encopresis |
| | Intestinal transit time decreased | Enteritis |
| | Irritable bowel syndrome | Enteritis leukopenic |
| | Neutropenic colitis | Enterocolitis |
| | Post-procedural diarrhea | Enterocolitis hemorrhagic |
| | Radiation proctitis | Eosinophilic colitis |

MeDRA = Medical Dictionary for Regulatory Activities;
SMQ = standardized MedDRA queries

Software

All data preparation and presentation was performed using SAS® Version 9.3 or later (SAS Institute, Cary, NC) and R Version 3.0.2. Parameter estimations and model simulations for pharmacokinetic analysis were completed using NONMEM version 7.3, with default setting to be FOCEI with Laplacian method. Package Perl Speaks NONMEM (PSN) version 3.7.6 was used for interface to NONMEM and for assessing models. R package Xpose4 version 4.5.0 was used to display results of model diagnostics.

Results

Efficacy was associated with longer duration of unencapsulated SN-38 (uSN38) above a threshold and higher C_{avg} of tIRI, tSN38 and uSN38. Neutropenia was associated with uSN38 Cₘₐₓ and diarrhea with tIRI Cₘₐₓ. Baseline predictive factors were race, BSA, and bilirubin.

Patients

Samples for pharmacokinetic measurements were collected during the first cycle of nal-IRI treatment in 5 Phase 1-2 studies and a Phase 3 study conducted in North America, Europe and Asia. Of the 368 treated patients, 353 (96%) had samples analyzed for pharmacokinetic measurement, including 97% (258/266) of patients in the Phase 3 study in metastatic pancreatic cancer (NAPOLI-1). Patient characteristics at baseline are listed in Table 6. Patients with hepatic or renal impairment were excluded from the enrollment; nevertheless, 20 patients were enrolled with bilirubin>1 mg/dL (19/20 had bilirubin between 1-2 mg/dL; 1 patient had bilirubin>2 mg/dL). The majority (73%) of the data was obtained from patients with metastatic pancreatic cancer. Most patients received an initial dose of 100 mg/m² (53%) or 70 mg/m² (39%). Most patients were either Caucasian (52%) or East Asian (42%).

TABLE 6
Patient Characteristics at Baseline (N = 353)

| | | | Median | |
|---|---|---|---|---|
| | | | (5^{th} and 95^{th} | |
| Characteristics | Subgroup | N (%)^{a} | Percentile) | |
| Sex | Female | 157 (44) | | |
| | Male | 196 (56) | | |
| Race | Caucasian | 182 (52) | | |
| | Others | 21 (6) | | |
| | East Asian | 150 (42) | | |
| Liver metastasis (for NAPOLI-1 | No | 87 (34) | | |
| only) | Yes | 171 (66) | | |
| Study name | NAPOLI-1 | 258 (73) | | |
| | Others | 95 (27) | | |
| UGT1A1*28 (for NAPOLI-1 | Non 7/7 | 244 (95) | | |
| only) | 7/7 | 14 (5) | | |
| Treatment (for NAPOLI-1 only) | nal-IRI + 5FU/LV | 116 (45) | | |
| | nal-IRI (mono) | 142 (55) | | |
| Tumor type at diagnosis | Colorectal cancer | 18 (5) | | |
| | Gastric & GEJ | 37 (10) | | |
| | cancer | | | |
| | Metastatic | 258 (73) | | |
| | pancreatic cancer | | | |
| | Solid tumor | 40 (11) | | |
| Initial dose, mg/m^{2,b} | 50 (60) | 4 (1) | | |
| | 70 (80) | 141 (40) | | |
| | 80 (90) | 6 (2) | | |
| | 90 (100) | 11 (3) | | |
| | 100 (120) | 187 (53) | | |
| | 150 (180) | 4 (1) | | |
| Age, y | | 353 | 63 | (39.8, 79.2) |
| Albumin, g/L | | 349 | 40 | (29, 47) |
| ALT, U/L | | 352 | 25 | (8.9, 96.3) |
| AST, U/L | | 352 | 29 | (14.7, 81.9) |
| Bilirubin (umol/L) | | 352 | 7 | (3, 19) |
| BSA, m² | | 353 | 1.7 | (1.3, 2.2) |
| CrCl, 10⁻³ L/s | | 352 | 1.36 | (0.66, 2.53) |

^{a}Percent only included in baseline characteristics with subcategories.
^{b}Dose is given based on irinotecan free base. The original protocol dose, based on irinotecan hydrochloride trihydrate, is in parentheses.
ALT = alanine aminotransferase;
AST = aspartate aminotransferase;
BSA = body surface area;
GEJ = gastroesophageal junction.

Pharmacokinetic Parameter Estimates

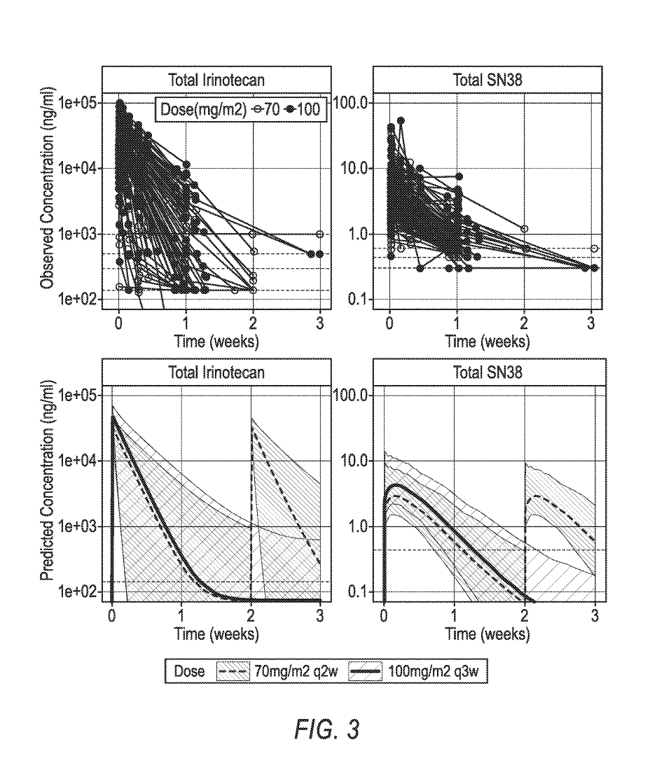

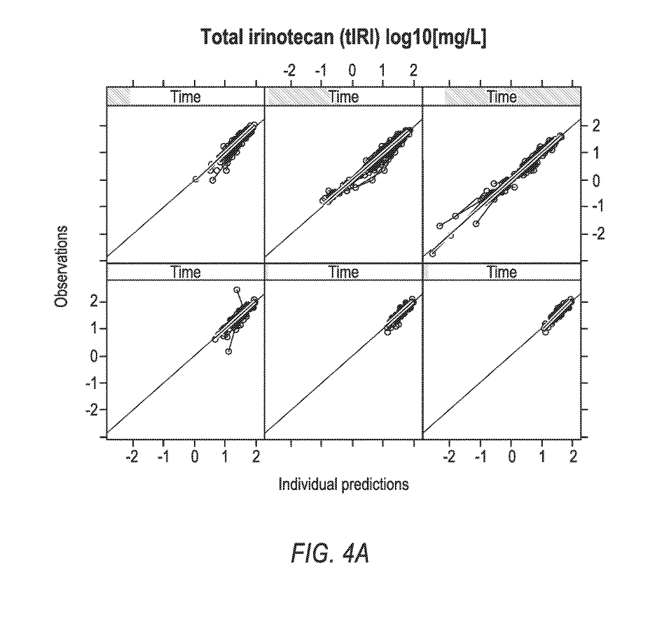

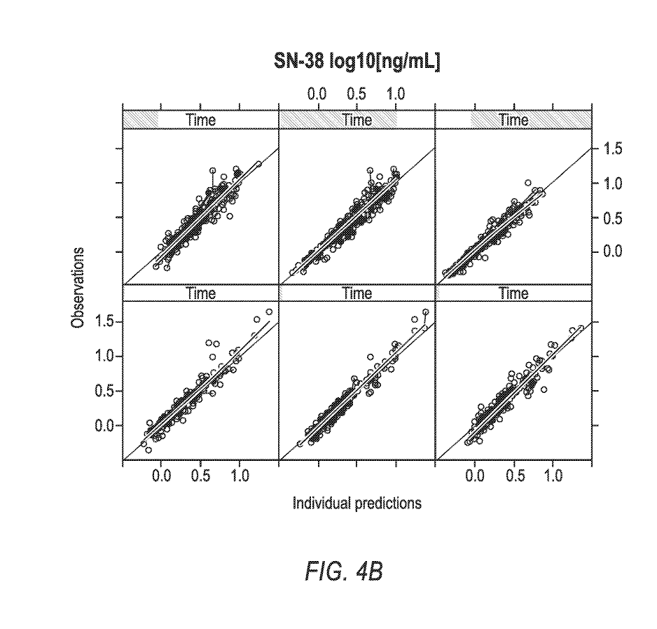

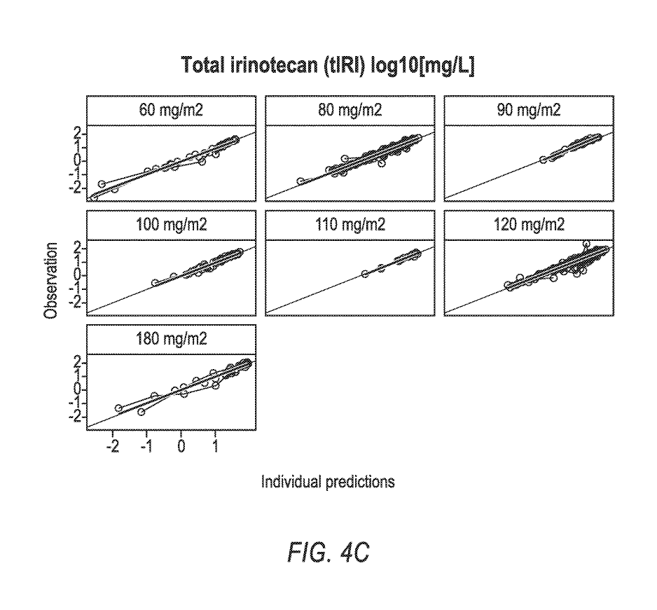

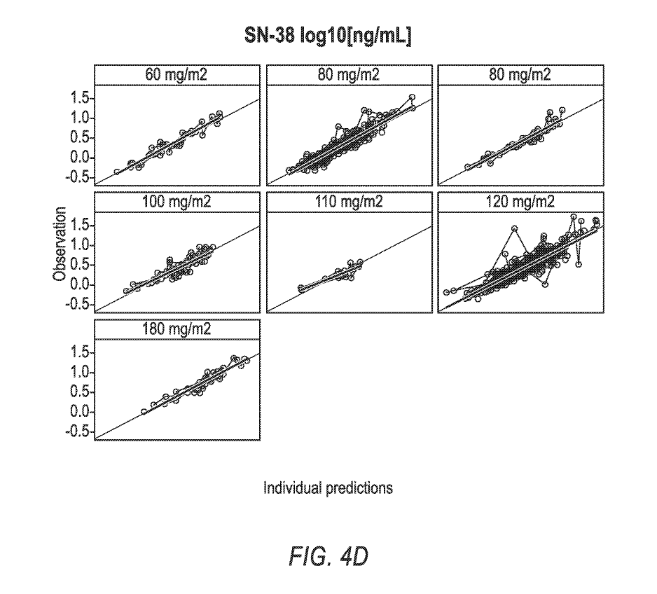

A total of 1,792 tIRI samples from 355 subjects and 1,765 tSN38 samples from 353 subjects were analyzed. Typical observed and predicted pharmacokinetic profiles with 70 mg/m² and 100 mg/m² are shown in FIG. 3. In the figure, the line represents the median prediction and the shaded areas are the 95% prediction intervals (q2w=administration Q2W; q3w=administration Q3W). The final model sufficiently described the data, as evidenced by the comparison of the data and model fits (FIGS. 4A-4D) and by visual predictive checks for the overall data, and stratified by dose. Final estimated parameters of IRI and SN-38 in the population pharmacokinetic models are listed in Table 7 and Table 8, respectively. The two measures of tSN38 and uSN38 were highly correlated (Kendall tau concordance were 0.81 and 0.70 for C_{avg} and Cₘₐₓ, respectively). The Cₘₐₓ and C_{avg} had low correlation for tIRI and uSN38 (Kendall tau concordance were 0.31 and 0.44, respectively).

TABLE 7
Final Estimated Parameters of Total Irinotecan (tIRI) Pharmacokinetics

| | | Estimated | Estimated Values from | | |
|---|---|---|---|---|---|
| | | Values | Bootstrapping (N = 497) | | |
| Parameter | Unit | (Final Model) | 50% | 2.5% | 97.5% |

| | | | | |
|---|---|---|---|---|
| Objective Function | −2184 | −2210 | −3210 | 16800 |

Fixed effects

| | | | | | |
|---|---|---|---|---|---|
| Volume (V1) | L | 4.60 | 4.58 | 4.14 | 4.99 |
| Clearance (CL) | L/week | 13.6 | 13.3 | 9.17 | 22.8 |
| Q | L/week | 0.471 | 0.47 | 0.114 | 1.55 |
| V2 | L | 48.7 | 48.0 | 46.7 | 49.4 |
| θ_{{V1,BSA}} | l/m² | 0.416 | 0.373 | 0.224 | 0.594 |
| θ_{{V1,mfg == NAPOLI}} | unitless (relative to | −0.172 | −0.198 | −0.362 | −0.0295 |
| | mfg = PEI) | | | | |
| θ_{{CL,race == Asian}} | unitless (relative to | 0.647 | 0.682 | 0.326 | 1.00 |
| | race = non-Asian) | | | | |
| θ_{{CL,treatment contain 5FU}} | unitless (relative to | 0.075 | 0.0592 | −0.152 | 0.248 |
| | treatment do not | | | | |
| | contain 5FU) | | | | |
| θ_{{CL,mfg == NAPOLI}} | unitless (relative to | −0.189 | −0.261 | −0.752 | 0.158 |
| | mfg = PEI) | | | | |
| θ_{{CL,liver metastasis}} | unitless (relative | −0.075 | −0.0511 | −0.341 | 0.157 |
| | no liver | | | | |
| | metastasis) | | | | |
| θ_{{CL,ALT}} | unitless (per unit | 0.016 | −0.143 | −0.640 | 0.399 |
| | change of log10 | | | | |
| | ALT) | | | | |
| θ_{{CL,albumin}} | unitless (per unit | −1.79 | −1.28 | −4.49 | 2.09 |
| | change of log10 | | | | |
| | albumin) | | | | |
| θ_{{CL,bilirubin}} | unitless (per unit | −0.0670 | 0.0510 | −0.454 | 0.608 |
| | change of log10 | | | | |
| | bilirubin) | | | | |

Random effects

| | | | | | |
|---|---|---|---|---|---|
| σ² (V1) | unitless (variance) | 0.068 | 0.0768 | 0.0276 | 0.156 |
| σ² (V1-CL)(off- | unitless (variance) | 0.184 | 0.176 | 0.0195 | 0.351 |
| diagonal) | | | | | |
| σ² (CL) | unitless (variance) | 0.843 | 0.871 | 0.503 | 1.52 |

Residuals

| | | | | | |
|---|---|---|---|---|---|
| σ² (in log10 | unitless (variance) | 0.038 | 0.035 | 0.013 | 0.081 |
| concentration) | | | | | |

The covariate structures followed Equation S1. For example, for CL, the form is provided below:

$${CL}_{j} = \hat{C} L exp ( {η}_{\{ CL , i \}} + {\sum }_{\{ i ∈ contcovset \}} {θ}_{\{ CL , i \}} ( {contcov}_{\{ ij \}} - {contcov}_{\{ i , median \}} ) + {\sum }_{\{ i ∈ contcovset \}} {θ}_{\{ CL , i \}} ( {catcov}_{\{ ij \}} ) )$$

ALT=alanine aminotransferase; FU=fluorouracil; tIRI=total irinotecan; mfg=manufacturing site; PEI=PharmaEngine studies; NA=not applicable.

TABLE 8
Final Estimated Parameters of SN-38 Pharmacokinetics

| | | Estimated | | | |
|---|---|---|---|---|---|
| | | Values | | | |
| | | (Final | Estimated Values from Bootstrap | | |
| Parameter Name | Units | Model) | Median | 2.5% | 97.5% |

| | | | | |
|---|---|---|---|---|
| Objective Function | −3117 | −3130 | −3520 | −2780 |

Fixed effects

| | | | | | |
|---|---|---|---|---|---|
| Clearance (CL_SN38) | L/week | 14.2 | 14.0 | 12.7 | 14.6 |
| Conversion flux rate | l/week | 0.00072 | 0.00075 | 0.00069 | 0.00079 |
| from irinotecan (Kcov) | | | | | |
| Fraction of SN38 per | ng (SN-38)/μg | 0.090 | 0.087 | 0.081 | 0.093 |
| unit of irinotecan | (irinotecan) | | | | |
| (fSN38) | | | | | |
| θ_{{CL}_{_{—}}_{SN38,race == Asian}} | unitless (relative to | −0.161 | −0.103 | −0.178 | −0.020 |
| | race == non Asian) | | | | |
| θ_{{CL}_{_{—}}_{SN38,UGT1A1 * 28 == homozygous}} | unitless (relative to | −1.46E−05 | −9.28E−06 | −1.14E−05 | −6.87E−06 |
| | UGT1A1*28 non- | | | | |
| | homozygous) | | | | |
| θ_{{CL}_{_{—}}_{SN38,treatment contains 5FU}} | unitless (relative to | −1.53E−04 | −1.46E−04 | −2.26E−04 | −6.62E−05 |
| | treatment do not | | | | |
| | contain 5FU) | | | | |
| θ_{{CL}_{_{—}}_{SN38,liver metastasis = YES}} | unitless (relative to | −0.002 | −0.002 | −0.004 | −0.001 |
| | no liver metastasis) | | | | |
| θ_{{CL}_{_{—}}_{SN38,ALT}} | unitless (per unit | −1.09E−05 | −1.24E−05 | −2.31E−05 | 1.26E−06 |
| | change of 10g10 | | | | |
| | ALT) | | | | |
| θ_{{CL}_{_{—}}_{SN38,albumin}} | unitless (per unit | −0.207 | −0.205 | −0.331 | −0.061 |
| | change of log10 | | | | |
| | albumin) | | | | |
| θ_{{CL}_{_{—}}_{SN38,bilirubin}} | unitless (per unit | −0.852 | −0.756 | −1.060 | −0.369 |
| | change of log10 | | | | |
| | bilirubin) | | | | |
| θ_{{CL}_{_{—}}_{SN38,CRCL}} | min/mL (per unit | −5.64E−05 | −6.46E−05 | −1.17E−04 | −8.87E−06 |
| | change of CrCL) | | | | |
| θ_{{Kcov,mfg == NAPOLI}} | unitless (relative to | 3.79E−05 | 4.55E−05 | 1.10E−05 | 7.80E−05 |
| | mfg == PEI) | | | | |
| θ_{{Kcov,tIRI}_{_{—}}_{logCL}} | unitless (per unit | 2.095 | 2.160 | 1.930 | 2.430 |
| | change of log10 tIRI | | | | |
| | CL) | | | | |
| θ_{{Kcov,tIRI}_{_{—}}_{logV1}} | unitless (per unit | −0.867 | −1.360 | −2.220 | −0.534 |
| | change of log10 tIRI | | | | |
| | V1) | | | | |

Random effects

| | | | | | |
|---|---|---|---|---|---|
| σ² (CL_SN38) | unitless (variance) | 0.155 | 0.140 | 0.114 | 0.154 |
| σ² (Kcov) | unitless (variance) | 0.184 | 0.198 | 0.173 | 0.227 |
| σ² (fSN38) | unitless (variance) | 0.500 | 0.497 | 0.379 | 0.549 |

Residuals

| | | | | | |
|---|---|---|---|---|---|
| σ² (in log10 | unitless (variance) | 0.021 | 0.021 | 0.016 | 0.026 |
| concentration) | | | | | |

The covariate structures followed Equation S1. For example, for CL, the form is provided below

$${CL}_{j} = \hat{C} L exp ( {η}_{\{ CL , i \}} + {\sum }_{\{ i ∈ contcovset \}} {θ}_{\{ CL , i \}} ( {contcov}_{\{ ij \}} - {contcov}_{\{ i , median \}} ) + {\sum }_{\{ i ∈ contcovset \}} {θ}_{\{ CL , i \}} ( {catcov}_{\{ ij \}} ) )$$

The estimated derived pharmacokinetic parameters are provided in Table 9. The estimated initial and terminal half-lives of tIRI were 38.2 (95% confidence interval [CI] 23.2-56.7) and 12200 (95% CI 3990-50200) hours; the terminal half-life of SN-38 was 38.2 (95% CI 36.5-41.9) hours. Compared to a nal-IRI dose of 100 mg/m² every 3 weeks, a dose of 70 mg/m² every 2 weeks was predicted to have similar tIRI and tSN38 C_{avg}, 1.5-fold lower tIRI and tSN38 Cₘₐₓ, and a similar tIRI Cₘᵢₙ but 7-fold higher tSN38 Cₘᵢₙ. tIRI was approximately 3-orders of magnitude higher than tSN38. The estimated volume was 4.58 L, a value comparable to typical blood volume.

TABLE 9
Summary of Irinotecan and SN-38 Pharmacokinetics Parameters by nal-
IRI dose Regimen in NAPOLI-1

| | Pharmacokinetic | 70 (80) mg/m² | 100 (120) mg/m² |
|---|---|---|---|
| Analyte | Parameter^{a} | Q2W^{b} | Q3W^{b} |
| Total irinotecan | C_{avg}, mg/L | 1.19 (0.91-1.55) | 1.66 (1.33-2.05) |
| | Cₘₐₓ, mg/L | 26.6 (24.1-29.3) | 41.5 (39.8-43.2) |
| | Clearance, L/week | 13.3 (9.17, 22.8) | |
| | Volume, L | 4.58 (4.14, 4.99) | |
| | First-phase t_{1/2}, h | 38.2 (23.2-56.7) | |
| | Terminal t_{1/2}, h | 12200 (3990-50200) | |
| Total SN-38 | C_{avg}, ng/mL | 0.721 (0.667-0.778) | 0.870 (0.821-0.922) |
| | Cₘₐₓ, ng/mL | 2.64 (2.47-2.83) | 3.99 (3.77-4.23) |
| | Clearance, L/week | 14.0 (12.7-14.6) | |
| | Terminal t_{1/2}, h | 38.2 (36.5-41.9) | |
| Unencapsulated | C_{avg}, ng/mL | 0.589 (0.543-0.639) | 0.702 (0.661-0.745) |
| SN-38 | Cₘₐₓ, ng/mL | 2.07 (1.93-2.23) | 3.05 (2.89-3.21) |
| | t_{uSN38>thr}, weeks (first 6 | 4.77 (4.59-4.95) | 4.28 (4.12-4.44) |
| | weeks, based on actual | | |
| | doses) | | |
| | t_{uSN38>thr}, weeks (first 6 | 5.71 (5.64-5.79) | 4.80 (4.69-4.92) |
| | weeks, based on simulated | | |
| | doses) | | |

C_{avg} = average concentration;
Cₘₐₓ = maximum concentration;
t_{uSN38>thr} = time uSN38>threshold
^{a}For C_{avg}, Cₘₐₓ, and t_{uSN38>thr}, median values and 95% prediction intervals (representing inter-patient variabilities) were obtained from NAPOLI1 patients; for Clearance, Volume, and t_{1/2}, median values and 95% confidence intervals (representing precision of parameter estimates) were obtained from bootstrapping.
^{b}Dose is given based on irinotecan free base. The original protocol dose, based on irinotecan hydrochloride trihydrate, is in parentheses.

Exposure-Efficacy Relationships

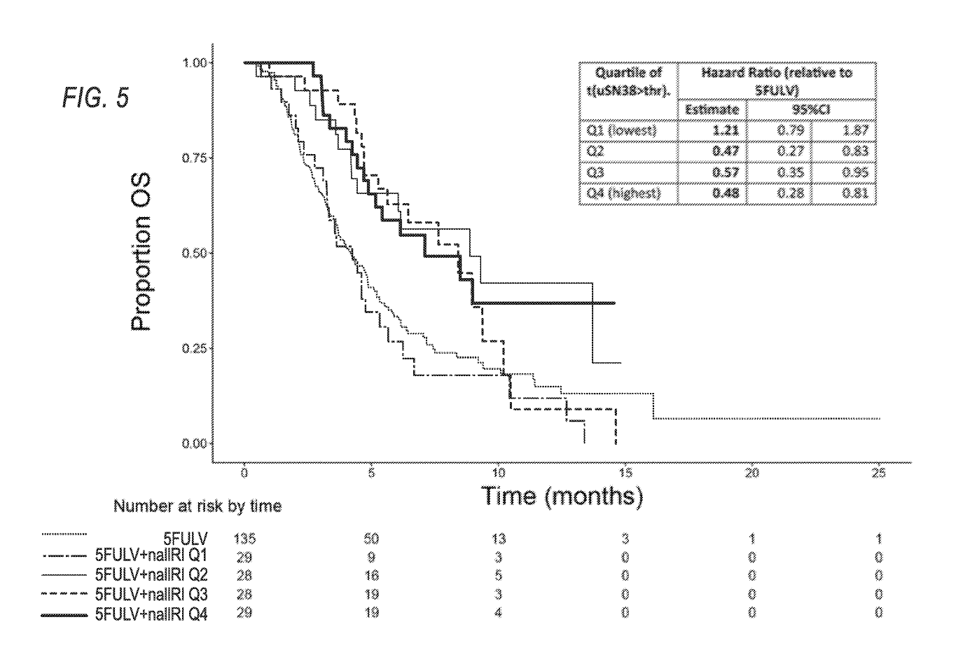

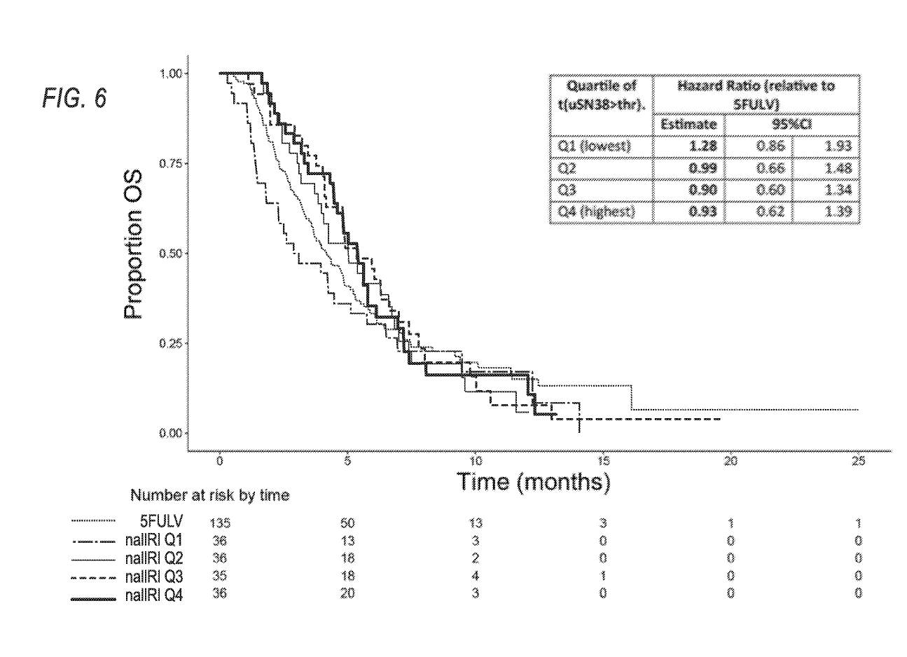

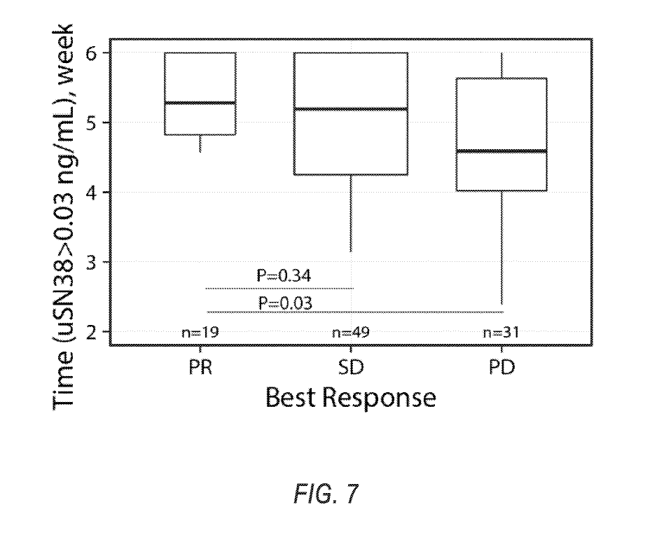

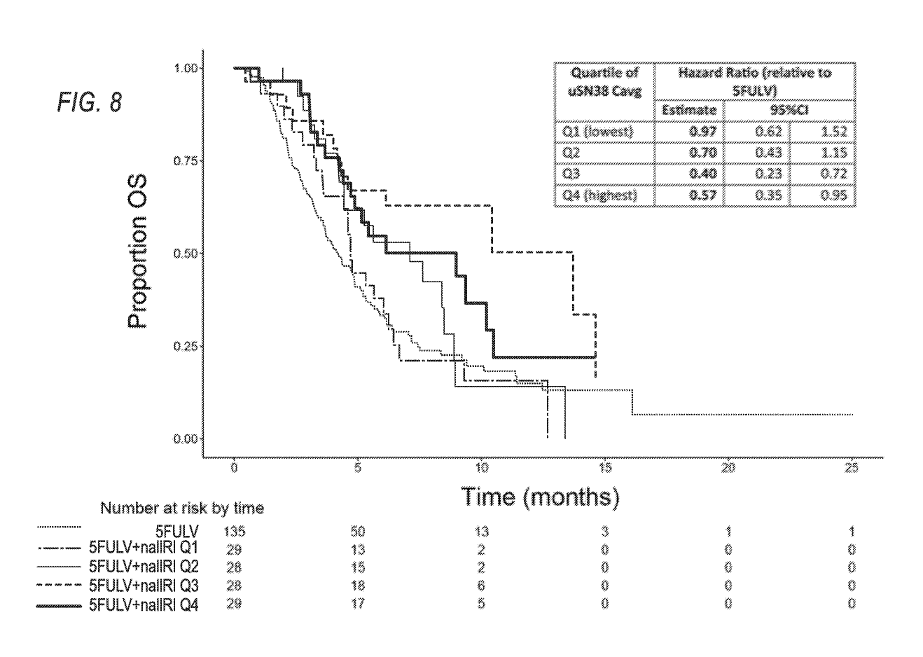

In the nal-IRI+5FU/LV arm of NAPOLI-1, longer overall survival (OS) and progression free survival (PFS) were associated with longer duration of uSN38 above threshold (duration_{uSN38>thr}) and higher C_{avg} of tIRI, tSN38 and uSN38, with the highest association observed for t_{uSN38>thr}. Cₘₐₓ of tIRI, tSN38, or uSN38 was not predictive of OS (P=0.58-0.98). The relationship between OS and quartiles of t_{uSN38>thr} for the nal-IRI 5-FU/LV and nal-IRI monotherapy arms are provided in FIG. 5 and FIG. 6, respectively. In the figures, CI=confidence interval; FU=fluorouracil; LV=leucovorin; OS=overall survival; and Q1-Q4 represent the quartiles of uSN38 time above threshold. Q1 represents the shortest time and Q4 represents the longest time. Longer t_{uSN38>thr} was associated with a higher probability of achieving objective response in the nal-IRI+5-FU/LV arm (FIG. 7). This association was not observed in the nal-IRI monotherapy arm. The association between OS and uSN38 C_{avg} is provided in FIG. 8, which also shows prolonged OS with higher uSN38 C_{avg} (uSN38 C_{avg} and t_{uSN38>thr} is correlated with Kendall τ of 0.48).

TABLE 10
Multivariate Analysis Cox Regression Model for OS (n = 257)

| | | | Risk Ratio (95% |
|---|---|---|---|
| Endpoint | Predictor | P | CI) |
| OS | duration_{uSN38>thr} | 0.0002 | 0.77 (0.68-0.89) |
| | (continuous variable) | | |
| OS | Coadministration with 5-FU/LV | 0.0323 | 0.72 (0.53-0.97) |
| | (categorical variable) | | |
| PFS | duration_{uSN38>thr} | 0.0002 | 0.77 (0.67-0.88) |
| | (continuous variable) | | |
| PFS | Coadministration with 5-FU/LV | 0.0417 | 0.73 (0.54-0.99) |
| | (categorical variable) | | |

Data: NAPOLI-1 nal-IRI monotherapy (n = 143) and nal-IRI + 5-FU/LV (n = 114) arms.
OS = overall survival;
PFS = progression-free survival.

Exposure-Safety Relationships

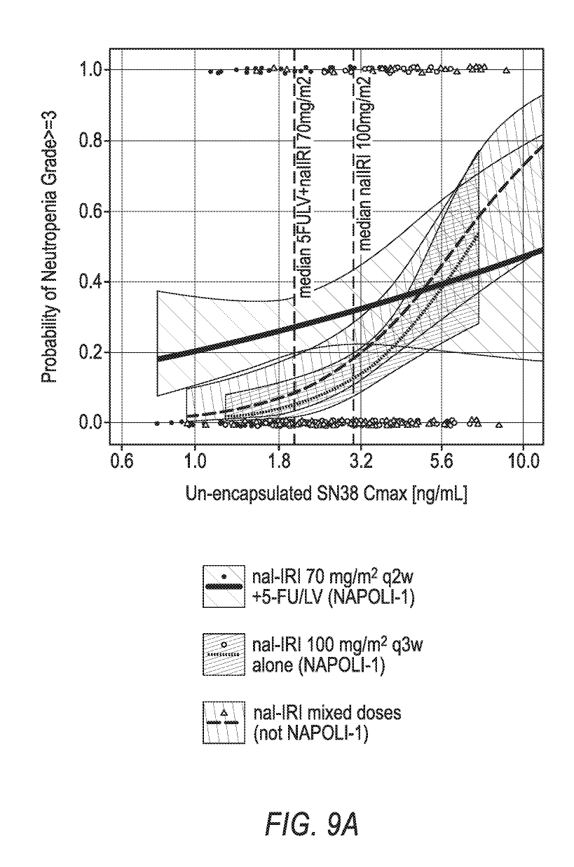

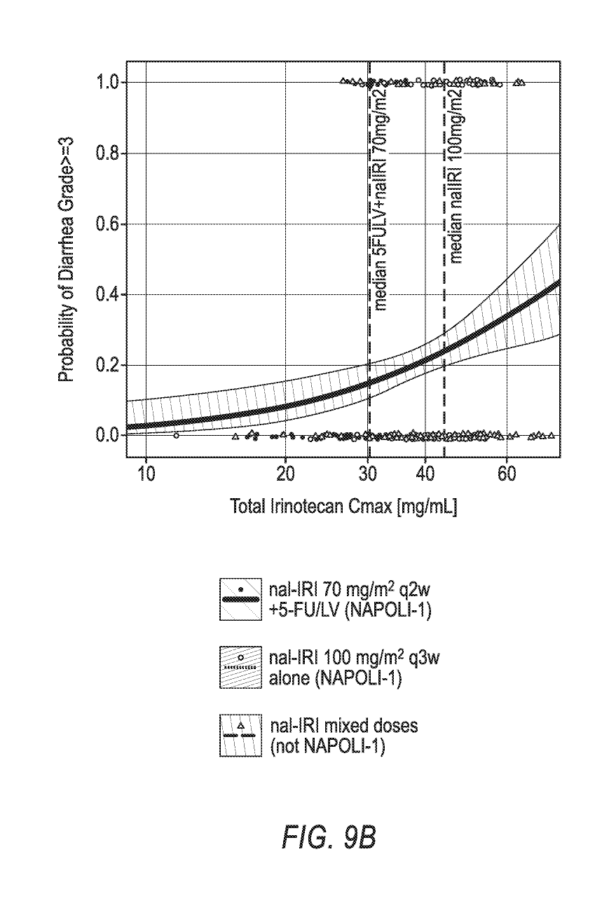

A total of 353 patients were included in the pharmacokinetics-safety analysis. Neutropenia was most strongly associated with uSN38 Cₘₐₓ (FIG. 9A). In the figure, CI=confidence interval; Cmax=maximum concentration; FU=fluorouracil; LV=leucovorin; q2w=administered Q2W; and q3w=administered Q3W. The solid lines in FIGS. 9A and 9B indicate the probability of incidence of adverse events, and the dotted lines represent median values for dose regimen of 70 mg/m² and 100 mg/m². Higher uSN38 concentrations were associated with a higher probability of both incidence and severity of neutropenia. The association with neutropenia was greater with the uSN38 than tSN38. For the incidence of neutropenia grade≧3, the association P-values were <0.001 and 0.08 for uSN38 and for tSN38, respectively. The association between uSN38 and neutropenia was also greater for Cₘₐₓ than for C_{avg} (e.g., grade≧3 neutropenia: P=<0.001 vs P=0.045 with uSN38 Cₘₐₓ and C_{avg}, respectively). In a multivariate logistic regression analysis of grade≧3 neutropenia (Table 11 a), the association between uSN38 and neutropenia was still significant (P=0.00005) even after adding factors known to predict neutropenia (baseline ANC and 5-FU/LV coadministration). When baseline factors predictive of uSN38 were included (race, bilirubin, and BSA), the association with uSN38 Cₘₐₓ was only borderline significant (P=0.068).

TABLE 11a
Multivariate Analysis Regression Results of Neutropenia Grade≧3

| | | Odds Coefficient | | |
|---|---|---|---|---|
| Model | Term | Estimate | SE | P-value |
| Model 1 (PK | (Intercept) | −1.24 | 0.66 | 0.0611 |
| parameter uSN38 | ANC | −3.16 | 0.79 | 0.00006 |
| Cₘₐₓ and known | 5-FU | 1.22 | 0.32 | 0.00011 |
| predictors of | coadministration = | | | |
| neutropenia) | TRUE | | | |
| | uSN38 Cₘₐₓ | 3.15 | 0.75 | 0.00003 |
| Model 2 (PK | (Intercept) | −1.91 | 1.71 | 0.2647 |
| parameter uSN38 | ANC | −2.67 | 0.82 | 0.0011 |
| Cₘₐₓ, known | 5-FU | 1.14 | 0.32 | 0.0004 |
| predictors of | coadministration = | | | |
| neutropenia, and | TRUE | | | |
| factors predictive of | uSN38 Cₘₐₓ | 1.58 | 0.86 | 0.0682 |
| SN-38) | Bilirubin | 1.39 | 0.74 | 0.0600 |
| | Race = Other | −0.86 | 1.07 | 0.4211 |
| | Race = Asian | 0.93 | 0.36 | 0.0091 |
| | BSA | −0.36 | 0.82 | 0.6653 |

Model 1 = obtained from stepwise selection;
Model 2 = using factors not predictive to uSN38.
ANC = absolute neutrophil count;
BSA = body surface area;
Cₘₐₓ = maximum concentration;
FU-fluorouracil;
SE = standard error;
uSN38 = unencapsulated SN-38.

Diarrhea was most strongly associated with tIRI Cₘₐₓ (FIG. 9B). Higher tIRI Cₘₐₓ was associated with a higher incidence and severity of diarrhea. The association was significant for grade≧3 diarrhea but not for grade≧1 diarrhea. The association between grade≧3 diarrhea and tIRI was greater for Cₘₐₓ (P=0.001) than for C_{avg} (P=0.019). The association between tIRI Cₘₐₓ and diarrhea was observed in both the Caucasian and Asian subpopulations. In NAPOLI-1, this association was observed within the nal-IRI monotherapy arm, but not within the nal-IRI+5FU/LV arm. This was likely due to the absence of patients with high tIRI Cₘₐₓ in the nal-IRI+5FU/LV arm and lower nal-IRI dose. In a multivariate logistic regression analysis of grade≧3 diarrhea (Table 11b), the identified predictive factors were tIRI Cₘₐₓ and race (Caucasian vs East Asian).

TABLE 11b
Multivariate Analysis Regression Results of Diarrhea Grade ≧3

| | | Odds Coefficient | | |
|---|---|---|---|---|
| Model | Term | Estimate | SE | P-value |
| Model 1 (final model from | (Intercept) | −5.38 | 1.95 | 0.01 |
| a stepwise feature | tIRI Cₘₐₓ | 3.90 | 1.14 | 0.0007 |
| selection) | race = Others | −1.06 | 0.78 | 0.17 |
| | race = Asian | −0.86 | 0.30 | 0.0040 |
| | ALT | −0.85 | 0.51 | 0.10 |
| | CrCl | −0.01 | 0.01 | 0.16 |

Liver metastasis status [values: −1 = not available and not in NAPOLI-1;
0 = no metastasis in NAPOLI-1;
1 = with metastasis in NAPOLI-1].
ALT = alanine aminotransferase;
CrCl = creatinine clearance.

Analysis of the NAPOLI-1 safety data showed that compared with Caucasian patients, East Asian patients who received nal-IRI+5-FU/LV had a higher incidence of NCI CTCAE Grade 3 or 4 neutropenia (55% [18/33] vs 18% [13/73], respectively), yet a lower incidence of Grade 3 or 4 diarrhea (19.2% [14/73] vs 3.0% [1/33], respectively.(20) Therefore, the differences in the observed rates of neutropenia and diarrhea by race can be explained by the racial differences in the Cₘₐₓ of tIRI and uSN38.

Baseline Factors Predictive of Plasma Pharmacokinetics

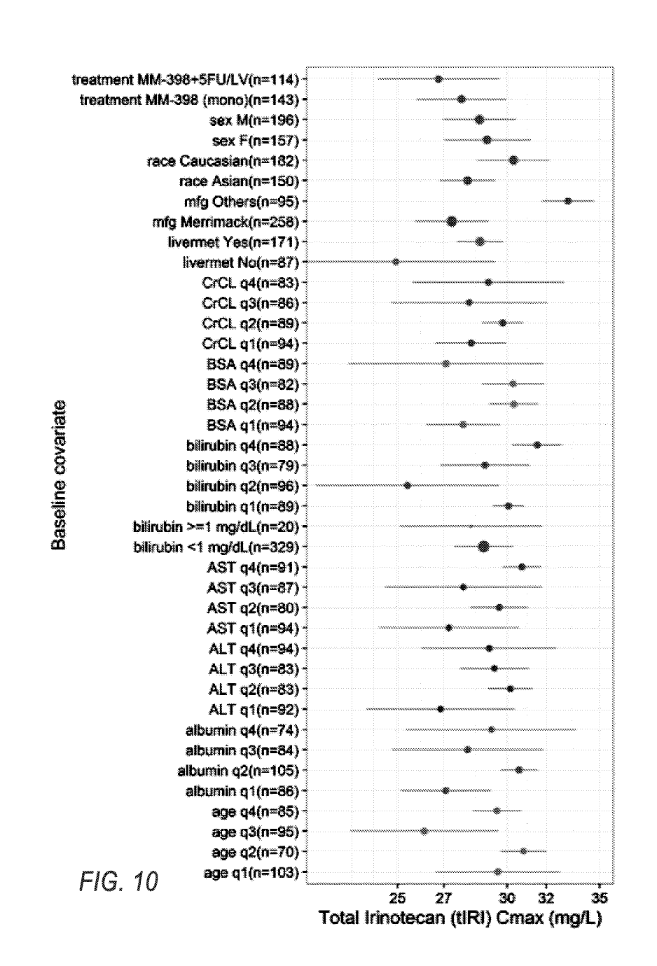

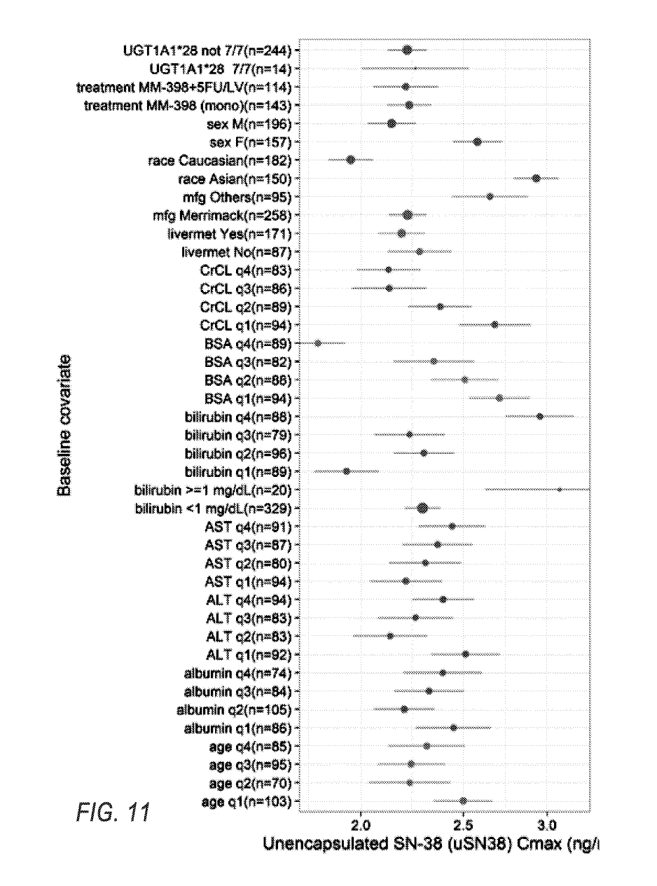

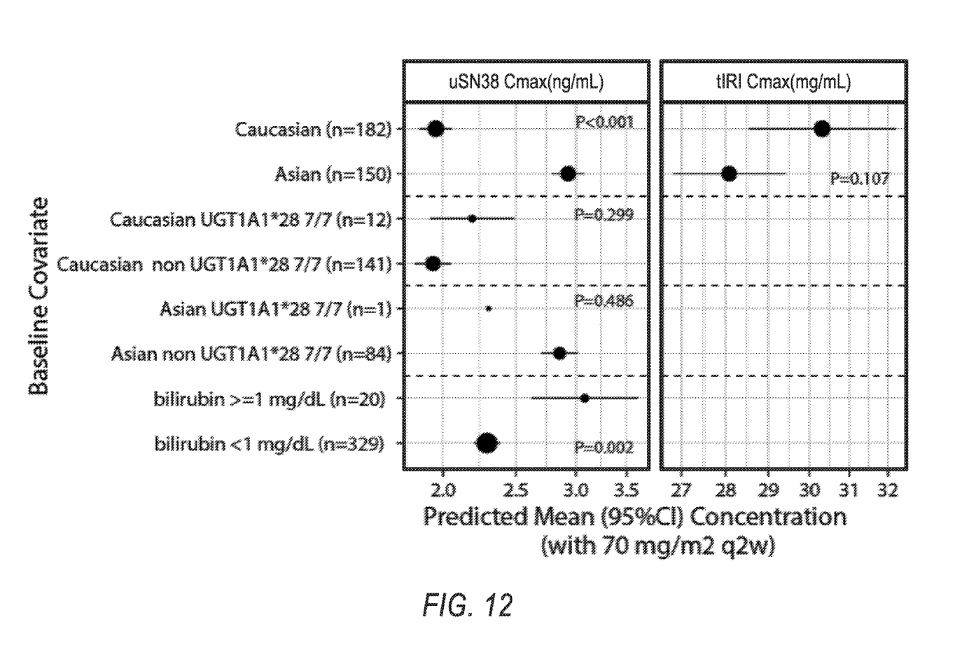

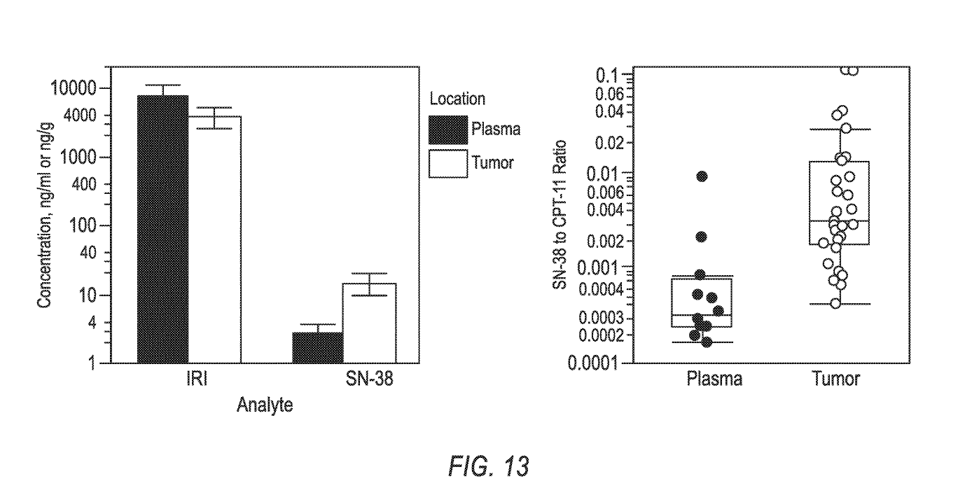

Baseline factors predictive of plasma pharmacokinetics were evaluated, including BSA, demographics, hepatic and renal function, pharmacogenomics (UGT1A1*28) and extrinsic factors (Table 3, FIG. 10, and FIG. 11). In the figures, points represent geometric means; whiskers represent 95% confidence intervals; and Pred Conc=predicted concentration. The significant factors and the corresponding concentrations of tIRI and uSN38 for nal-IRI 70 mg/m² are summarized in FIG. 12. In the figure, CI=confidence interval, and q2w=administered Q2W.

TABLE 12
Comparison of Simulated Pharmacokinetic Parameters for Fixed Dosing
and BSA-based Dosing Strategies (N = 353)

| | | | | Median | |
|---|---|---|---|---|---|
| | | | Geometric | (25th %, | |
| | Pharmacokinetic | | Mean ± SD | 75th % | |
| Analyte | Parameter | Dose Strategy | (log-scale) | Quartile) | IQR/Median, % |
| tIRI | C_{avg} | BSA-based | 1.90 (0.68) | 3.15 (1.79-5.43) | 116% |
| | C_{avg} | Fixed | 1.97 (0.66) | 3.18 (1.74-5.28) | 111% |
| | Cₘₐₓ | BSA-based | 28.81 (0.23) | 30.68 (27.15-33.85) | 22% |
| | Cₘₐₓ | Fixed | 29.07 (0.19) | 30.26 (26.67-34.38) | 25% |
| uSN38 | C_{avg} | BSA-based | 0.83 (0.2) | 0.81 (0.63-1.05) | 51% |
| | C_{avg} | Fixed | 0.83 (0.21) | 0.81 (0.62-1.07) | 57% |
| | Cₘₐₓ | BSA-based | 2.32 (0.19) | 2.28 (1.75-2.99) | 55% |
| | Cₘₐₓ | Fixed | 2.32 (0.21) | 2.26 (1.69-3.25) | 69% |

Simulation was performed using 70 mg/m² for BSA-based strategy or 116.7 mg for fixed dosing strategy (equivalent to 70 mg/m² dosing for a median BSA). BSA=body surface area; IQR=interquartile range; tIRI=total irinotecan; uSN38=unencapsulated SN-38.

Factors with significant association with tIRI pharmacokinetics were race and BSA. Factors with significant association with tSN38 were race, BSA, and bilirubin. Asians had lower tIRI and higher uSN38 compared with Caucasians (7% and 78% lower tIRI Cₘₐₓ and c_{avg}, 50% and 20% higher uSN38 Cₘₐₓ and C_{avg}; all P≦0.001). In the population pharmacokinetics model that accounted for multivariate analysis (including BSA), race remained a significant factor for both tIRI and tSN38 (Table 7 and Table 8). Comparison of BSA-based dosing to fixed dosing (70 mg/m² or an equivalent fixed dose of 116.7 mg) revealed that BSA-based dosing reduced variability of tIRI and uSN38 Cₘₐₓ (3% and 14% less interquartile range, Table 12). This result implies a benefit of BSA-based dosing in reducing the variability of tIRI and uSN38 Cₘₐₓ. While the number of patients with elevated bilirubin was small (n=20), bilirubin was found to be a significant factor of tSN38: compared with patients with bilirubin<1 mg/dL, patients with bilirubin≧1 mg/dL had a higher uSN38 C_{avg} (43% higher) and Cₘₐₓ (35% higher).

UGT1A1*28, a pharmacogenomic biomarker, was not a significant predictor of SN-38 with nal-IRI administration. In the population pharmacokinetics dataset, the prevalence of UGT1A1*28 7/7 homozygosity in Asians was low (2/129 [1.5%]). Compared with non-7/7 homozygous Caucasians, 7/7 homozygous Caucasians had similar uSN38 Cₘₐₓ if both were dosed at 70 mg/m² (FIG. 12; 2.19 [95% CI 1.92-2.49, n=12] and 1.94 [95% CI 1.84-2.05, n=141] ng/mL; P=0.30; geometric mean ratio: 1.13 [95% CI 0.90-1.42]. In NAPOLI-1, the actual dose homozygous patients received were lower than the dose in nonhomozygous patients). The estimated SN-38 clearance in UGT1A1 7/7 homozygous was 1.000-times (0.0% difference) the clearance in non-7/7 (Table 8). A sensitivity analysis was performed to estimate the SN-38 clearance by more detailed categories of UGT1A1*28 alleles (separate evaluation for 6/6, 6/7, and 7/7; Table 13). The estimated clearance for UGT1A1*28 6/7 and UGT1A1*28 7/7 were within 0.0% and 2.7% of the clearance for UGT1A1*28 6/6. These results indicate that UGT1A1 is not a significant covariate to SN-38 clearance.

TABLE 13
SN-38 Pharmacokinetic Model Parameter Estimates with Covariates for
UGT1A1*28 Specific Alleles

| | | |
|---|---|---|
| | | Estimated |
| Parameter Name | Unit | Values |

| | |
|---|---|
| Objective Function | −2637 |

Fixed effects

| | | |
|---|---|---|
| Clearance (CL_SN38) | L/week | 1.41E+01 |
| Conversion flux rate from irinotecan (Kcov) | L/week | 7.51E−02 |
| Fraction of SN38 per unit of irinotecan (fSN38) | ng (SN-38)/μg | 9.41E−02 |
| | (irinotecan) | |
| θ_{{CL}_{_{—}}_{SN38,race == Asian}} | unitless (relative to | −1.84E−01 |
| | race = non-Asian) | |
| θ_{{CL}_{_{—}}_{SN38,UGT1A1 * 28 == 7/7}} | unitless (relative to | 6.68E−06 |
| | UGT1A1*28 6/6) | |
| θ_{{CL}_{_{—}}_{SN38,UGT1A1 * 28 == 6/7}} | unitless (relative to | 2.74E−02 |
| | UGT1A1*28 6/6) | |
| θ_{{CL}_{_{—}}_{SN38,treatment contains 5FU}} | unitless (relative to | 8.11E−04 |
| | treatment do not | |
| | contain 5FU) | |
| θ_{{CL}_{_{—}}_{SN38,liver metastasis = YES}} | unitless (relative to no | 7.17E−03 |
| | liver metastasis) | |
| θ_{{CL}_{_{—}}_{SN38,ALT}} | unitless (per unit | −8.52E−06 |
| | change of log10 | |
| | ALT [IU/mL]) | |
| θ_{{CL}_{_{—}}_{SN38,albumin}} | unitless (per unit | 9.24E−02 |
| | change of log10 | |
| | albumin [g/dL]) | |
| θ_{{CL}_{_{—}}_{SN38,bilirubin}} | unitless (per unit | −5.88E−01 |
| | change of log 10 | |
| | bilirubin [umol/L]) | |
| θ_{{CL}_{_{—}}_{SN38,CRCL}} | min/mL (per unit | 2.95E−04 |
| | change of CrCL) | |
| θ_{{Kcov,mfg == PEI}} | unitless (relative to | 3.86E−05 |
| | mfg == NAPOLI) | |
| θ_{{Kcov,tIRI}_{_{—}}_{logCL}} | unitless (per unit | 1.90 |
| | change of tIRI CL) | |
| θ_{{Kcov,tIRI}_{_{—}}_{logV1}} | unitless (per unit | 4.61E−01 |
| | change of tIRI V1) | |
| θ_{{Kcov,BSA}} | 1/m² (per unit change | −1.07 |
| | of BSA) | |
| θ_{{fSN38,mfg == PEI}} | unitless (relative to | −7.07E−01 |
| | mfg == NAPOLI) | |

Random effects

| | | |
|---|---|---|
| σ² (CL_SN38) | unitless (variance) | 8.74E−02 |
| σ² (Kcov) | unitless (variance) | 1.62E−01 |
| σ² (fSN38) | unitless (variance) | 3.85E−01 |

Residuals

| | | |
|---|---|---|
| σ² (in log10 concentration) | unitless (variance) | 2.38E−02 |

ALT = alanine aminotransferase;
FU = fluorouracil;
IRI = irinotecan;
NA = not applicable.
Fold-change is specified as fold-change relative to the typical values of each parameter. Bold rows indicate the estimates by specific UGT1A1*28 alleles.

The covariate structures followed Equation S1. For example, for CL, the form is provided below.

$${CL}_{j} = \hat{C} L exp ( {η}_{\{ CL , i \}} + {\sum }_{\{ i ∈ contcovset \}} {θ}_{\{ CL , i \}} ( {contcov}_{\{ ij \}} - {contcov}_{\{ i , median \}} ) + {\sum }_{\{ i ∈ contcovset \}} {θ}_{\{ CL , i \}} ( {catcov}_{\{ ij \}} ) )$$

^{b}The dataset for this run is different than the dataset of the overall population pharmacokinetic dataset. In this run, only patients with detailed UGT1A1 information were included.

Other baseline factors evaluated were found not to have significant associations with tIRI or uSN38. Among measures of hepatic functions other than bilirubin, albumin had a weak association with tIRI, but not tSN38 nor uSN38). Moreover, the direction of the association was opposite of that expected in patients with hepatic impairment and opposite of the observation of diminished clearance of irinotecan reported in patients with hepatic impairment administered with free irinotecan. Because of the lack of association with the active metabolite SN-38, the effect of albumin is unlikely to be clinically relevant. Sex and creatinine clearance were also not significantly associated with SN-38 after adjusting for BSA.

Discussion

Liposomal encapsulation with nal-IRI extends the half-lives of irinotecan and SN-38. The association between SN-38 exposures and efficacy supports the potential benefit of nal-IRI in maintaining extended SN-38 concentrations to achieve optimal antitumor activity.

Similar to the liposomal formulation of doxorubicin, the liposomal formulation of irinotecan modifies pharmacological properties of irinotecan, resulting in extended half-lives of plasma total irinotecan and SN-38. The extended plasma pharmacokinetics observed with nal-IRI provides a tool to distinguish C_{avg} and Cₘₐₓ, as evidenced by the low correlation between the two concentrations that is useful to evaluate pharmacological properties predictive of efficacy and safety. The vastly different estimated volumes highlight the different disposition characteristics with liposomal formulation.

In pancreatic cancer patients treated with nal-IRI+5-FU/LV, higher C_{avg} and longer duration_{uSN38>thr} was associated with longer OS and PFS and higher ORR. Conversely, Cₘₐₓ was not associated with OS. This is consistent with the hypothesis that dividing cells are sensitive to chemotherapy, thus prolonged duration of chemotherapy drug exposures allow greater number of tumor cells to be affected. The observed association between C_{avg} and longer duration_{uSN38>thr} with efficacy indicates a strong association between plasma and tumor concentrations. This is also supported by the direct SN-38 measurements in biopsies during a phase 1 trial that demonstrate increased tumor SN-38 pharmacokinetics with nal-IRI administration. Furthermore, the association between these 2 parameters and efficacy is consistent with the preclinical finding that showed that the in vivo activity of nal-IRI was strongly associated with the duration of exposure of SN-38 above a minimum inhibitory concentration. This result indicates the potential benefit in extending duration of plasma and tumor exposure via liposomal encapsulation.

Neutropenia and diarrhea are the most prominent adverse events with nal-IRI treatment. For neutropenia, unencapsulated SN-38 was the analyte that has the highest association, with Cₘₐₓ exhibiting a stronger association than C_{avg}. The association between neutropenia and uSN38 Cₘₐₓ appeared to be robust and remained significant in the presence of known factors predictive of neutropenia (e.g., ANC and 5-FU coadministration). Diarrhea was associated with total irinotecan Cₘₐₓ, and as was seen with neutropenia, the association was stronger with Cₘₐₓ than C_{avg}. The dichotomization of the analytes associated with blood and gut-related safety events are consistent with reports of differential metabolism occurring in the plasma and in the gut. In particular, it has been reported that SN-38G can be converted back to SN-38 in the gut via microflora, but this mechanism is absent in the plasma. Because SN-38G in the plasma is observed at an approximately 10-times higher concentration than SN-38, the conversion in the gut may result in higher SN-38 concentrations in the gut compared with the plasma. While the ratio of SN-38 and SN-38G would depend on the activity of UGT enzymes, the sum of SN-38 and SN-38G—including that in the gut—would increase as total drug exposure of irinotecan increased. As total drug exposure of nal-IRI is linearly proportional to plasma tIRI, it can be hypothesized that plasma tIRI is a surrogate measurement of the sum of SN-38 and SN-38G in the gut lumen.

Among the baseline factors considered, race (Caucasian vs East Asian) was the most significant predictive factor for both plasma total irinotecan and SN-38 pharmacokinetics following the administration of nal-IRI. Specifically, when compared with Caucasian patients, East Asian patients had lower tIRI and higher SN-38 concentrations, and a lower corresponding risk for diarrhea and higher risk for neutropenia. The race-pharmacokinetics association has not been reported in patients receiving non-liposomal irinotecan. Therefore, the release kinetics of irinotecan from liposome maybe linked to the race-related pharmacokinetic difference. The elimination of liposomal chemotherapy from circulation was hypothesized to follow two pathways: passive leakage from liposomes and active uptake by mononuclear phagocyte system (MPS). The passive leakage is likely to be dependent on external factors such as manufacturing. Therefore, race may affect the active uptake by MPS and provides direction for future research in exploring pharmacogenomic factors.

The levels of plasma SN-38 depend on both the incoming load of SN-38 and the activity of UGT enzymes. The activity of UGT enzymes can be assessed by either baseline bilirubin or by pharmacogenomics (UGT1A1*28). Liposomal encapsulation appears to reduce the incoming load of SN-38 by controlling the release of irinotecan. Hyperbilirubinemia, a surrogate of reduced UGT activity, has been shown to be predictive to plasma SN-38 and to neutropenia with administration of non-liposomal irinotecan. In patients administered with nal-IRI described here, baseline bilirubin was also found to be a significant predictor of SN-38, and SN-38 concentrations were 44% higher in patients with hyperbilirubinemia. Because of the limited number of patients with bilirubin>1 mg/dL in the dataset, no nal-IRI dose recommendation is provided, and a lower starting dose may be warranted.

Consistent result are found by pharmacogenomics (UGT1A1*28). In patients treated with non-liposomal irinotecan, the associations between UGT1A1*28 7/7 homozygosity and hematological toxicity were observed only in patients treated with doses>150 mg/m²; however, similar hematological toxicities were observed for both UGT1A1*28 homozygous and non-homozygous patients with a lower dose of non-liposomal irinotecan of 100-125 mg/m² every week. The association between UGT1A1*28 7/7 homozygosity and SN-38 concentrations are also dependent on the dose of non-liposomal irinotecan, with much higher SN-38 concentrations observed for 6/7 and 7/7 (compared to 6/6) when irinotecan was administered at a dose of 300 mg/m² than when it was administered at a dose of 15-75 mg/m² daily for 5 days for 2 consecutive weeks. With nal-IRI treatment, SN-38 pharmacokinetics were similar across UGT1A1*28 polymorphisms. A likely mechanistic explanation is that the liposomal encapsulation protects the majority of irinotecan from being converted into SN-38 and, therefore, the slow release of irinotecan allows the lower load of SN-38 to be metabolized by UGT enzymes even in patients with reduced UGT enzyme activities (for example, UGT1A1*28 7/7 homozygous patients). Additional data in Phase 1-2 studies in patients treated with nal-IRI tested for different UGT1A1 genotypes (UGT1A9*22 (*1b), UGT1A1G-3156A, UGT1A1*6, UGT1A1*27, UGT1A1T-3279G and DPYD*2A) indicate that no difference in SN-38 concentrations was observed by UGT1A1 genotypes. Because of the lack of precision in the comparison between homozygous and nonhomozygous patients (as evidence by the wide 95% CI range of the ratio), the limited number of patients homozygous for the UGT1A1*28 allele treated with nal-IRI, and the lower starting nal-IRI dose used in NAPOLI-1 for these patients (50 mg/m²), it is recommended that those known to be homozygous for the UGTIA1*28 allele be treated initially with 50 mg/m², which can be increased to 70 mg/m² if tolerated. However, UGT1A 1*28 testing is not mandated.

In conclusion, the quantification of the plasma pharmacokinetics in patients treated with nal-IRI showed the benefit of the liposomal formulation in extending the half-lives of irinotecan and SN-38. The differential pharmacological parameters associated with efficacy and safety endpoints provide support to the selection of dose regimen for nal-IRI. Because efficacy is associated with C_{avg} and duration_{uSN38>thr}, and safety is associated with Cₘₐₓ, a dose regimen of 70 mg/m² every 2 weeks would result in improved safety while maintaining efficacy as compared to a dose regimen of 100 mg/m² every 3 weeks. Therefore, these associations support the benefit in the current dosing of nal-IRI of 70 mg/m² every 2 weeks.

Texts and Models

General Structure and Assumptions of the Pharmacokinetic Model with nal-IRI Administration

The PK model aims to describe the two analyte measurements: total irinotecan (tIRI) and total SN-38 (tSN38). The general structure of the model is provided in FIG. 1. The two models are sequential: tIRI model is independent and tSN38 depends on the results of tIRI model. The independency of the tIRI model is justified by the four orders of magnitude difference in plasma concentration of tIRI and tSN38, therefore, the conversion rate from IRI to SN-38 is negligible compared to the tIRI clearance rate.

Mechanistically, encapsulated irinotecan (eIRI) is expected to be released from the liposome, and the unencapsulated IRI (uIRI) is subsequently converted to unencapsulated SN-38 (uSN38). However, the ratio of eIRI:tIRI was observed to be constant over one week of measurement in study PEP0201 (n=121 matched tIRI and eIRI samples from 11 patients, a slope of log₁₀(eIRI:tIRI ratios) by time of −0.000026 h⁻¹). Therefore, a model simplification was made to represent in one-step the release of tIRI to uIRI and the conversion of uIRI to uSN38.

The proposed model assumed that distribution of uIRI to peripheral tissues to be negligible with nal-IRI administration. Theoretically, the uIRI could undergo both metabolisms (to uSN38 and other metabolites) and distribution to peripheral tissues as reported with the administration of non-liposomal irinotecan (Chabot, et al., Annals Oncol 6: 141-151 1995; Xie et al, Journal of Clinical Oncology, Vol 20, No 15 (Aug. 1), 2002: pp 3293-3301). However, the kinetics of uIRI is expected to be rate-limited by the liposome clearance (estimated tIRI half-life of 38.2 is appropriate.

Total SN-38 is assumed to be a sum of both encapsulated SN-38 (eSN38) and un-encapsulated SN-38 (uSN38). The uSN38 is formed from tIRI, with a first order rate constant of formation. The eSN38 is assumed to be proportional to tIRI, with a time-invariant ratio. The underlying assumption is that the eSN38 is a contaminant of the administered nal-IRI, and at any given PK sample, eSN38 is present as a fraction of the measured nal-IRI quantity (quantified as eIRI concentration, which is approximately 95% of tIRI concentration and eIRI is in time-invariant ratio to tIRI). Only uSN38 is eliminated with a first order rate constant that is proportional to the concentration of uSN38. The assumption of no metabolism for eSN38 is supported by the data in patients that showed the absence of metabolite SN-38G (glucuronidated form of SN-38) in the first 12 hours after nal-IRI administration (see FIG. 2).

Final Pharmacokinetic Model of Total Irinotecan with nal-IRI Administration

The PK model of total irinotecan is a two-compartment model with first order elimination. The basic parameter is central volume (V1), central clearance (CL), peripheral volume (V2) and inter-compartmental clearance (Q). The inter-patient variability of parameters in the PK model is modeled as proportional (e.g., for CL):

$${CL}_{j} = \hat{C} L exp ( {η}_{\{ CL , i \}} + {\sum }_{\{ i ∈ contcovset \}} {θ}_{\{ CL , i \}} ( {contcov}_{\{ ij \}} - {contcov}_{\{ i , median \}} ) + {\sum }_{\{ i ∈ contcovset \}} {θ}_{\{ CL , i \}} ( {catcov}_{\{ ij \}} ) )$$

where j is subject, CLⱼ is the clearance for subject j, ĈL is the population estimate of CL, contcov_{{ij}} the continuous covariate i of subject j, catcov_{{ij}} is the categorical covariate i of subject j, and θ_{{CL,i}} is the estimate of the relationship between CL and covariate i across population. The parameter (CL, V1)-covariate relationship is pre-specified in Table 5. Parameter V2 and Q are estimated as fixed effects, without interpatient variability. Residual variability is modeled as additive in the log-scale of the concentration.

Final PK Model of Total SN-38 with nal-IRI Administration

Total SN-38 is represented as the sum of encapsulated SN-38 and un-encapsulated SN-38. The encapsulated SN-38 assumes to be a proportion of the total irinotecan concentration, with a proportionality constant follows a proportional relationship fⱼ=\hat{f}exp [symbol] (n_{{f,j}}) where fⱼ is the proportionality constant for subject j, and \hat{f} is the population estimate of f. The un-encapsulated SN-38 is modeled as a one compartmental model with input as conversion from total irinotecan and output as clearance (FIG. 1). The basic PK parameters include input rate constant (Kin), clearance (CL), and central volume (V). The inter-patient variability of parameters in the PK model is modeled as proportional (e.g., for CL):

$${CL}_{j} = \hat{C} L exp ( {η}_{\{ CL , i \}} + {\sum }_{\{ i ∈ contcovset \}} {θ}_{\{ CL , i \}} ( {contcov}_{\{ ij \}} - {contcov}_{\{ i , median \}} ) + {\sum }_{\{ i ∈ contcovset \}} {θ}_{\{ CL , i \}} ( {catcov}_{\{ ij \}} ) )$$

where j is subject, CLⱼ is the clearance for subject j, ĈL is the population estimate of CL, contov_{{ij}} is the continuous covariate i of subject j, catcov_{{if}} is the categorical covariate i of subject j, and θ_{{CL,i}} is the estimate of the relationship between CL and covariate i across population. The parameter (Kin, CL)-covariate relationship is pre-specified in Table 7. In the SN-38 model, tIRI clearance is included as a baseline covariate of SN-38 formation because tIRI clearance is a surrogate for the activity of the mononuclear phagocyte system (MPS) which may affect the metabolism and formation of SN-38. Parameter V is estimated as a fixed effect without interpatient variability. Residual variability is modeled as additive in the log-scale of the concentration.

1. MM-398 has a half-live of 16-27 h for total irinotecan and 49-57 h for SN-38.

2. Direct measurement of liposomal irinotecan shows that 95% of irinotecan remains liposome-encapsulated.

3. MM-398 is largely confined to vascular fluid:

Vd of MM-398 [80 mg/m² (salt)]: 2.2 L/m²

(Vd of Camptosar®: 110-234 L/m² (1))

TABLE 14
Median (% IQR)* Total Irinotecan and SN-38 PK Parameters in Patients
with Solid Tumors (dosing reported as the salt form of MM-398)

| MM-398 | Total Irinotecan | | | | SN-38 | | |
|---|---|---|---|---|---|---|---|
| Dose | Cₘₐₓ | T_{1/2} | AUC_{0-∞} | V_{d} | Cₘₐₓ | T_{1/2} | AUC_{0-∞} |
| (mg/m²) | (μg/mL) | (h) | (h · μg/mL) | (L/m²) | (ng/mL) | (h) | (h · ng/mL) |
| 80 | 38.0 | 26.8 | 1030 | 2.2 | 4.7 | 49.3 | 587 |
| (N = 25) | (36%) | (110%)^{a} | (169%)^{a} | (55%)^{a} | (89%) | (103%)^{b} | (69%)^{b} |
| 120 | 59.4 | 15.6 | 1258 | 1.9 | 7.2 | 57.4 | 574 |
| (N = 45) | (41%) | (198%) | (192%) | (52%) | (57%) | (67%)^{c} | (64%)^{c} |

% IQR: % Interquartile Ratio = (Interquartile-range)/Median * 100%;
T_{1/2}, AUC_{0-∞}, and V_{d} were calculated only for a subset of patients with sufficient number of samples in the terminal phase: ^{a} n = 23, ^{b} n = 13, and ^{c} n = 40.

Comparison of PK MM-398 120 mg/m² (salt) vs Camptosar® 300 mg/m² showed:

1. Total irinotecan: higher exposure (Cmax 13.4-fold, AUC0-∞46.2-fold)

2. SN-38:higher t1/2 and AUC0-∞ (t1/2 3.0-fold, and AUCO-∞1.4-fold); lower Cmax (0.19-fold)

TABLE 15
Comparison of PK MM-398 120 mg/m² (salt) vs Camptosar ® 300 mg/m²

| | | | MM-398 | Camptosar ® |
|---|---|---|---|---|
| | Parameter | Unit | 120 mg/m² | 300 mg/m² |
| Total | Cₘₐₓ | μg/mL | 55.2 | 4.1 |
| Irinotecan | | | (48.2-63.3) | (3.7-4.6) |
| | AUC_{0-∞} | h · μg/mL | 1140.9 | 24.7 |
| | | | (799.6-1628.0) | (21.5-28.4) |
| | T_{1/2} | h | 14.0 | 7.1 |
| | | | (10.3-19.2) | (6.2-8.2) |
| SN-38 | Cₘₐₓ | ng/mL | 7.1 | 37.5 |
| | | | (5.9-8.6) | (30.0-46.9) |
| | AUC_{0-∞} | h · ng/mL | 591.2 | 409.1 |
| | | | (465.8-750.3) | (348.5-480.1) |
| | T_{1/2} | h | 63.7 | 20.8 |
| | | | (50.3-80.5) | (17.7-24.5) |

Study CITS (n=13) measured concentration in patients tumor biopsies at 72 h after administration of MM-398 at 80 mg/m² (salt) showed: 1. Higher SN-38 in tumor than in plasma (4-times); and 2. Higher ratio of SN-38: irinotecan in tumor vs in plasma (8-times).

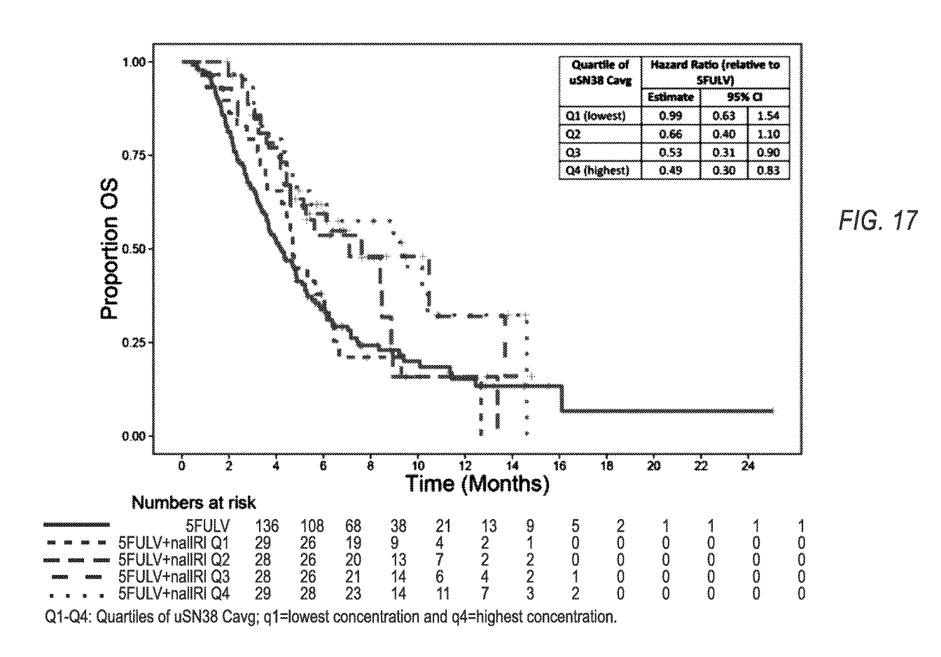

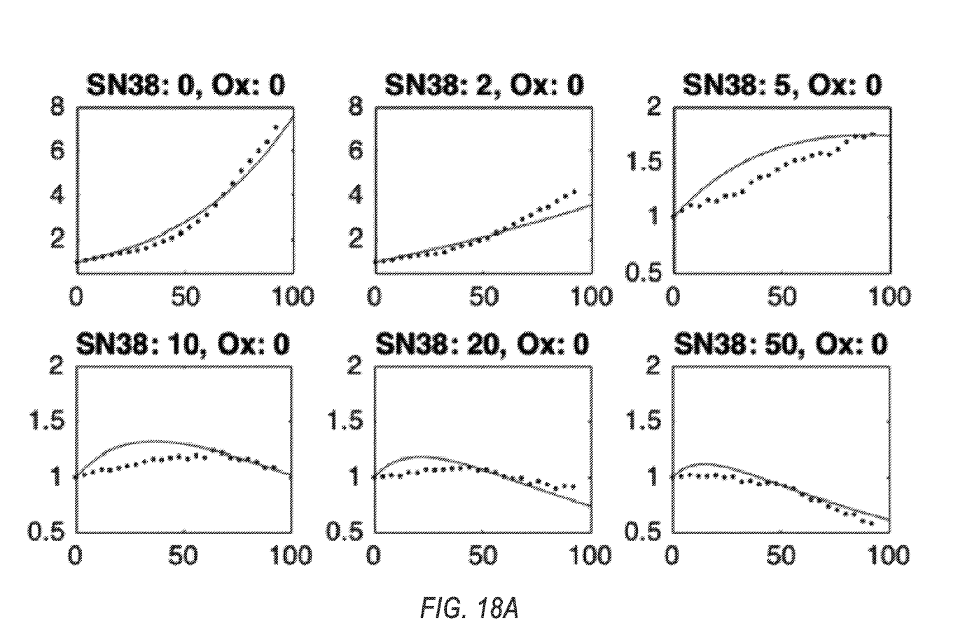

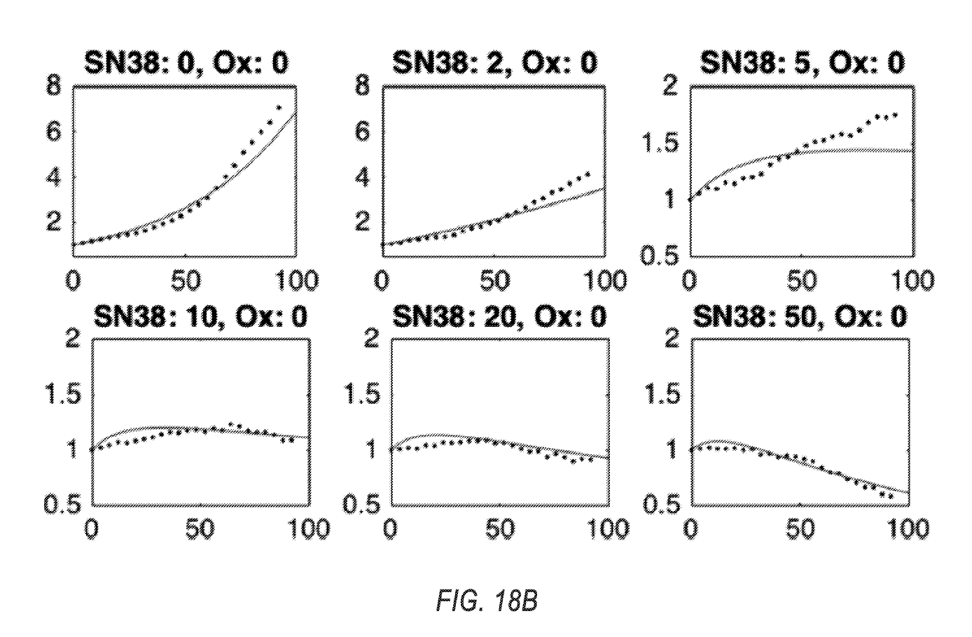

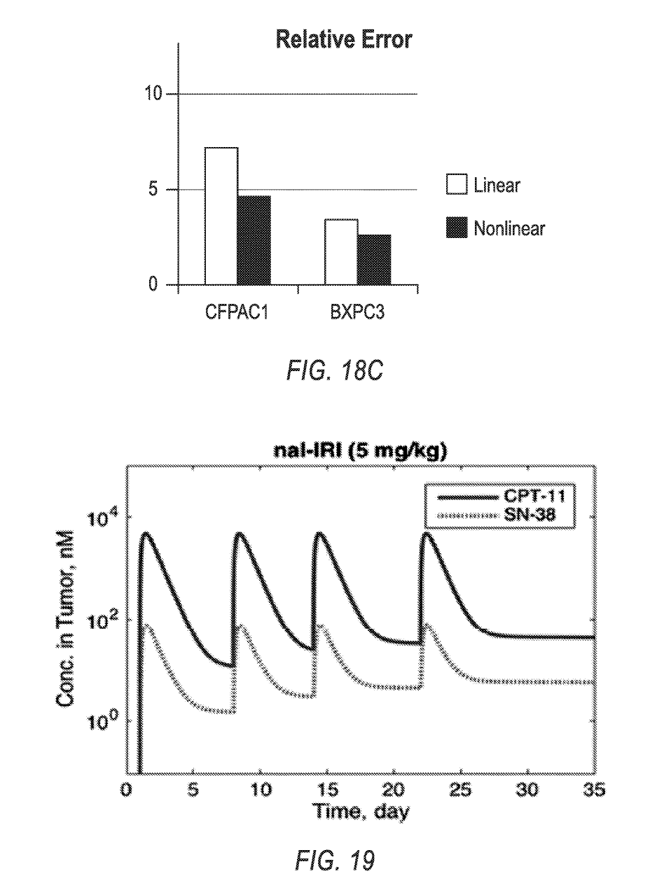

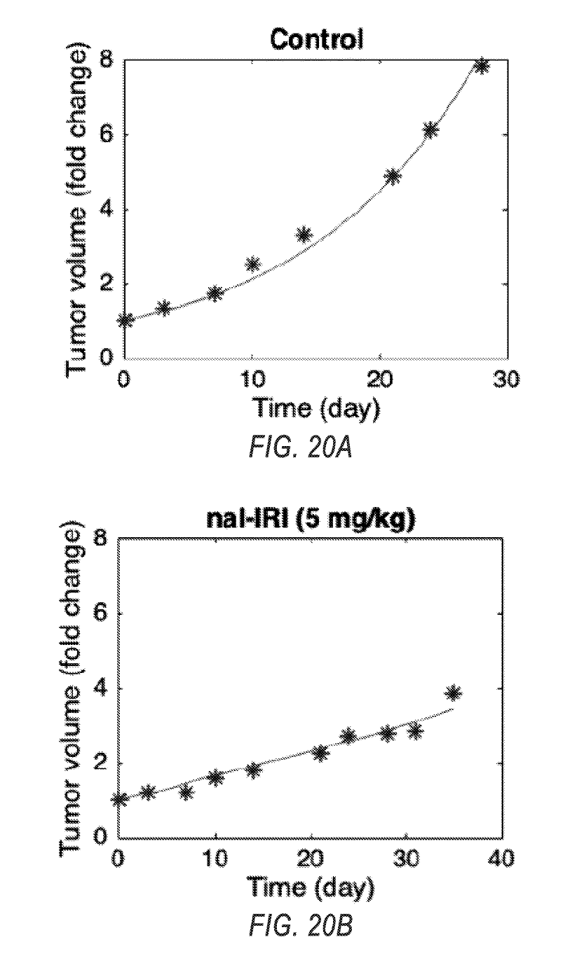

Both MM-398 and free irinotecan activity can be estimated from SN-38 tumor duration and SN-38 AUC in tumor is not predictive of in vivo activity (FIG. 17).

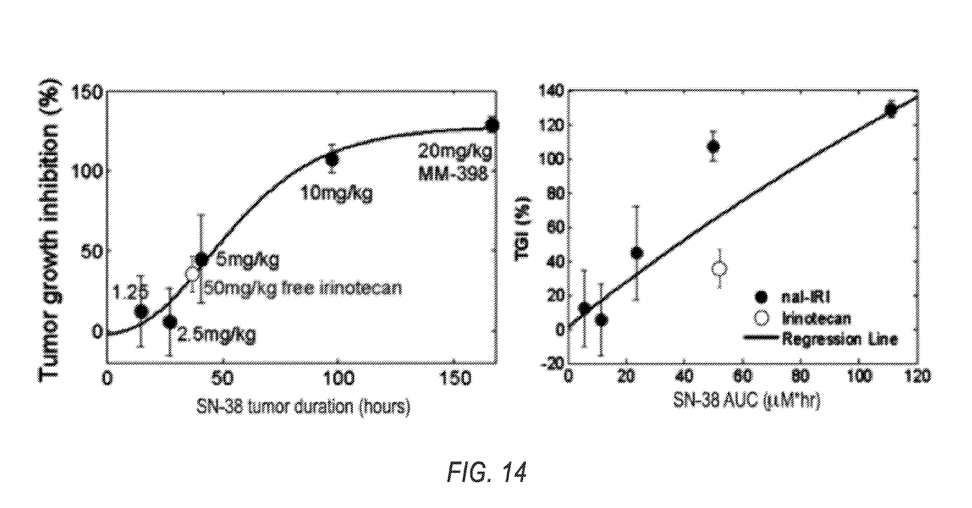

Hybrid tumor PK model supported 80 mg/m² (salt) q2w. Q2W 80 mg/m² (salt) could increase the duration of SN-38 levels in tumor compared to equal exposure Q3W 120 mg/m² (salt) (FIG. 14).

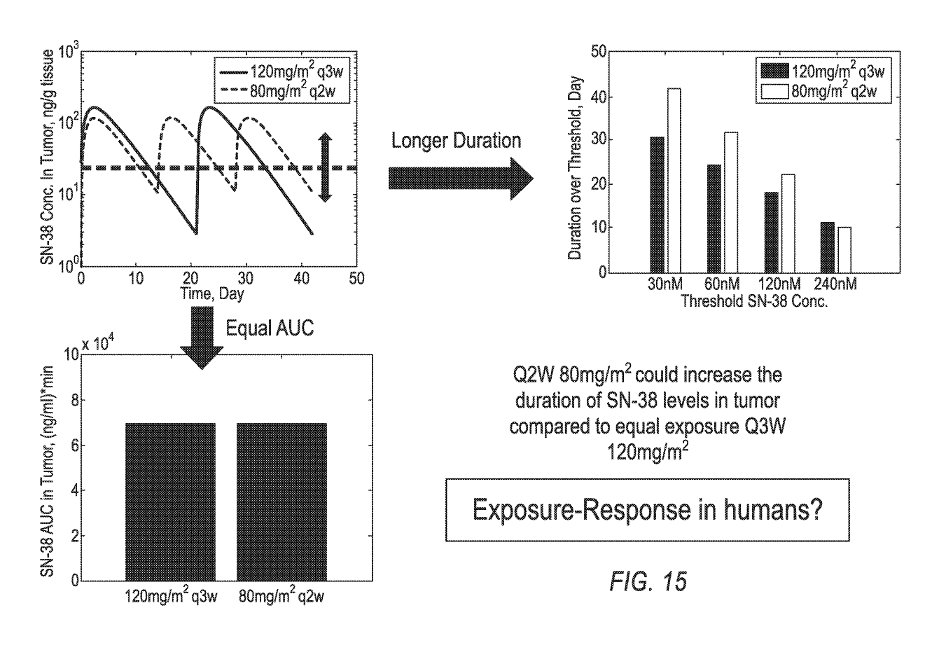

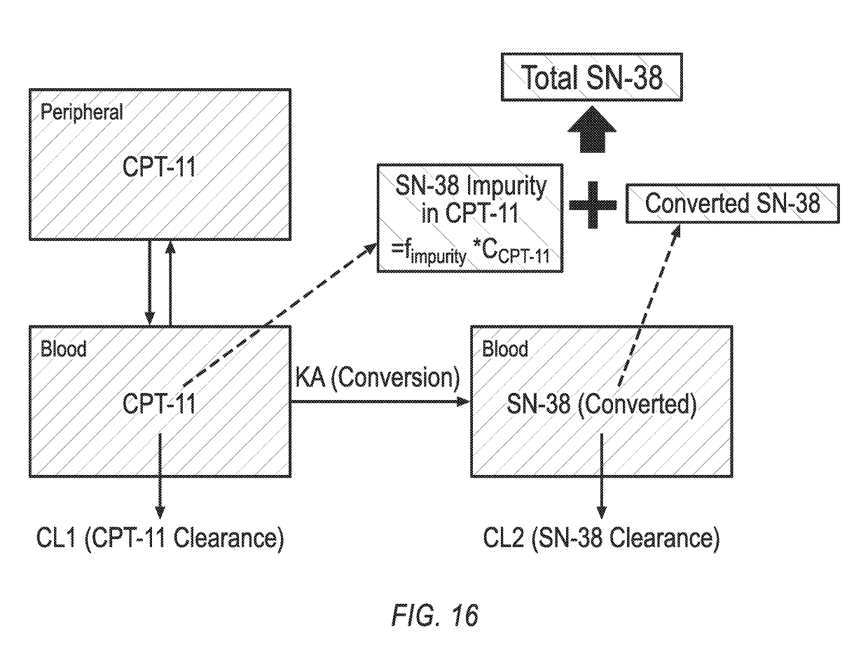

No exposure-response relationship and high inter-patient variability. Cmaxs for CPT-11 and SN-38, and AUCi for CPT-11 were proportional to MM-398 dose and AUCi for SN-38 was not proportional to MM-398 dose. Inter-patient variability was greater than the dose effect (FIG. 15).

Population PK analysis:

Understand quantitative relationship among drug concentrations, patient characteristics and safety/efficacy responses.

Identify factors that affect drug behavior or explain variability in a target population.

Sparse PK sampling can be used for late stage studies, e.g. in NAPOLI-1, 2˜3 time points per patient.

Covariate model structure, Table 17:

TABLE 17
Covariate model structure

| Covariates | Parameters | Rational and References |
|---|---|---|
| BSA | Volume | Body size, MM-398 was dosed per |
| | | BSA |
| Age, sex, and race | Clearance | Standard covariates |
| Manufacturing site | Clearance | Potential difference in |
| | | manufacturing of MM-398 in |
| | | phase 2 and phase 3 studies |
| Co-administration | Clearance | Effect of co-administration |
| with 5-FU | | |
| AST, ALT, bilirubin, | Clearance | Hepatic function |
| albumin, and liver | | |
| metastasis | | |
| Creatinine clearance | Clearance | Renal function |

Comparison of MM-398 and Camptosar PK profiles suggests not all SN-38 are bioavailable from MM-398.

SN-38 impurity in MM-398 could contribute to the early SN-38 peak signal (non-bioavailable SN-38): ˜0.01% of total CPT-11 in MM-398.

Since average Cmax from 120 mg/m² MM-398 (salt) was ˜60 ug/ml, it could generate>˜6 ng/ml of SN-38 impurity which is close to SN-38 Cmax (˜8 ng/ml) from MM-398

Max allowable SN-38 impurity in free irinotecan: <0.15%

Most of SN-38 in early time points following MM-398 could be SN-38 impurity (liposomal)

Population PK, PK-Efficacy, and PK-Safety of MM-398 (n=353) (Table 17)

Total irinotecan and SN-38 exposure from MM-398 were simulated to establish exposure-response relationships from NAPOLI-1 study.

TABLE 17
Population PK, PK-Efficacy, and PK-Safety of MM-398 (n = 353)

| | PK parameters |
|---|---|
| Endpoint | with the highest association |
| Efficacy (OS, PFS, ORR) (inNAPOLI1 | SN-38 Total¹ C_{average} |
| MM-398 + 5FU/LV arm) | SN-38 Converted² C_{average} |
| Safety: Neutropenia | SN-38 Converted Cₘₐₓ |
| Safety: Diarrhea | Total irinotecan³ Cₘₐₓ |
| Safety: Anemia | SN-38 Converted Cₘₐₓ |

¹SN-38 Total: the sum of encapsulated and un-encapsulated SN-38
²SN-38 Converted: the un-encapsulated SN-38 originating from the in vivo conversion of released irinotecan (model predicted)
³Total irinotecan: the sum of encapsulated and un-encapsulated irinotecan

Fixed vs. BSA based dosing (Table 18):

SN-38 Converted Cmax variability is lower with BSA-based dosing

TABLE 18
Fixed vs. BSA based dosing

| PK | Dose | | Geometric | | | Inter Quartile |
|---|---|---|---|---|---|---|
| Parameters | Strategy | N | Mean | SD (log scale) | Median | Range/Median |
| C_{avg} | BSA-based | 353 | 0.74 | 0.20 | 0.71 | 56% |
| C_{avg} | Fixed | 353 | 0.74 | 0.22 | 0.73 | 59% |
| Cₘₐₓ | BSA-based | 353 | 2.13 | 0.19 | 2.08 | 57% |
| Cₘₐₓ | Fixed | 353 | 2.13 | 0.22 | 2.08 | 74% |

TABLE 19
Simulated and observed 5-FU PK

| | Steady-state Conc. (mg/L) | | | Predicted 6-week Average AUC | | | | | |
|---|---|---|---|---|---|---|---|---|---|
| | GLS Mean | | Ratio² | AUC (h mg/L) | | | % AUC>20 h mg/L | | |
| Reference for | | MM398 + | Mean | | MM398 + | | | MM398 + | |
| 5-FU PK Parameters | 5FULV | 5FULV | [95% CI] | 5FULV | 5FULV | Ratio² | 5FULV | 5FULV | Diff³ |
| Bressolle 1999 | 1.091 | 0.683 | 0.626 | 17.456 | 15.710 | 0.9 | 41% | 35% | −6% |
| Mueller 2013 | 0.901 | 0.564 | 0.626 | 14.418 | 12.976 | 0.9 | 10% | 4% | −6% |
| Bressolle 1999 + | 1.212 | 0.759 | 0.626 | 19.392 | 17.453 | 0.9 | 49% | 42% | −7% |
| NAPOLI-1 5-FU | | | | | | | | | |
| concentration | | | | | | | | | |
| Woloch 2012 | 2.771 | 1.735 | 0.626 | 44.329 | 39.896 | 0.9 | 96% | 94% | −2% |
| NAPOLI-1 observed | 0.22 | 0.14 | 0.63 | | | | | | |
| 5-FU concentration | | | | | | | | | |

¹The majority (75%) of the 5-FU concentrations were collected after the end of infusion, therefore the observed concentration is lower than the steady-state concentration (5-FU was cleared rapidly after the end of infusion with the estimated half-life of 8-14 minutes).
²Ratio is defined as concentration or AUC ratio of MM-398 + 5-FU/LV relative to 5-FU/LV
³Difference is defined as percentage of MM-398 + 5-FU/LV minus percentage for 5-FU/LV

Comparison of Efficacy by different 5-FU Doses in Colorectal Cancer

1. Meta-analysis of studies directly comparing 5-FU doses in Colorectal Cancers.

2. Ranges of 5-FU dose intensities (425-2600 mg/m²/week for bolus, 800-2400 mg/m²/week for continuous infusion) are much larger than the difference in 5FU dose in NAPOLI-1 arms (1200 vs 1333 mg/m²/week).

3. Different 5-FU dose intensities and dose administrations did not affect OS (HR ranged 0.96-1.11).

BL=bolus; CI continuous infusion; nrd=not reached; ref=reference

TABLE 20
Comparison of Efficacy by different 5-FU Doses in Colorectal Cancer

| | | 5-FU Dose Regimen | | | | Overall Survival | | | | |
|---|---|---|---|---|---|---|---|---|---|---|
| | Study | Dose (mg/m² or mg/m²/d) | | Dose Intensity | | | | Median | OS | OS |
| Ref | Name | [infusion duration in h] | Adm | (mg/m²/week) | N | HR | P | OS (m) | 3 y | 5 y |
| Kohne | PET | 370-425 mg/m²/d for 5 d q4w | BL | 463-531 | 804 | 0.96 | 0.74 | nrd | 85% | 79% |
| 2013 | ACC-2 | (1) 3500 mg/m² [48 h] q1w | CI | 3500 | 797 | ref | | nrd | 85% | 79% |
| | | (2) 2600 mg/m² [24 h] q1w | | 2600 | | | | | | |
| | | (3) 400 mg/m² BL + 600 mg/m² | | 800 | | | | | | |
| | | [22 h] for 2 d q2w | | | | | | | | |

Dose Finding Methods: 3+3 vs CRM

Simulation to compare MTD dose-finding methods based on MM398 data

Result: higher likelihood for correct recommended MTD

Summary:

1. Evidence of MOAs via PD markers

2. Design of clinical pharmacology studies:

PK collections (optimal PK sampling selection)

3. Dose-finding methods (dose-pk-efficacy/safety)

MTD:more accurate dose finding with CRM (vs 3+3)

Multiple doses tested in efficacy study

4. Dose recommendations in subpopulations

Based on Population PK and exposure(PK)-efficacy/safety

5. Meta-analysis (dose-efficacy-safety)

Nanoliposomal irinotecan (nal-IRI, MM-398, PEP02) is irinotecan encapsulated in liposome nanoparticles designed to prolong circulation, enhance delivery, and increase conversion of irinotecan to SN-38 in tumors. In a study evaluating plasma pharmacokinetics (PK) of nal-IRI 120 mg/m² (salt) and irinotecan HCl 300 mg/m² (FIG. 2), nal-IRI resulted in longer half-lives and higher total irinotecan (tIRI), average (Cavg), and maximum (Cmax) concentrations, while maintaining a lower SN-38 Cmax. In a Phase 3 study in patients with metastatic pancreatic cancer previously treated with gemcitabine (NAPOLI-1), nal-IRI 80 mg/m² (salt) every 2 weeks in combination with 5-fluorouracil and leucovorin (5-FU/LV) was shown to extend overall survival (OS) compared with 5-FU/LV alone.

The objectives of this study were to quantify plasma population PK of nal-IRI in patients with, to understand the association between baseline covariates and plasma PK, and to evaluate the association between plasma PK with safety (diarrhea and neutropenia) and with the efficacy endpoints in patients with metastatic pancreatic cancer previously treated with gemcitabine (NAPOLI-1 population).

FIG. 2 is a comparison of plasma PK in patients treated with nal-IRI (n=37) or with irinotecan HCl (n=27). Comparing nal-IRI to irinotecan HCl, total irinotecan AUC was 46 times greater, total irinotecan Cmax was 13.4 times greater, SN-38 AUC was 1.4 times greater, and SN-38 Cmax was 0.19 times greater.

Population pharmacokinetic analysis of nal-IRI was performed for plasma concentrations of tIRI and its metabolite SN-38 (tSN38) in 353 patients across 6 studies (Table 2). The un-encapsulated SN-38 (uSN38) concentration was predicted from the model and appears to be the active metabolite (a fraction of SN-38 was encapsulated inside the liposome and is not bioavailable). SN-38G is the glucuronidated metabolite of SN-38 and is inactive.

PK-safety association was evaluated in a pooled dataset of 353 patients for the most significant adverse-events: neutropenia and diarrhea. PK-efficacy association was evaluated for OS, progression-free survival (PFS) and objective response rate (ORR) in patients from NAPOLI-1.

Patient Characteristics (N=353)

Patient characteristics at baseline are listed in Table 6. The median age was 63; 56% male; 52% Caucasian and 42% Asian. Patients with hepatic or renal impairment were excluded from the enrollment; 20 patients had bilirubin>1 mg/dL (19/20 had bilirubin between 1-2 mg/dL; 1 patient had bilirubin>2 mg/dL). The majority (73%) of the data was obtained from patients with metastatic pancreatic cancer. The majority had an initial dose of 120 mg/m² (salt) (53%) or 80 mg/m² (salt) (40%).

A total of 1,800 tIRI samples (355 subjects) and 1,773 tSN38 samples (353 subjects) were analyzed. The time-course of tIRI concentrations were modeled as a two-compartmental model. The time-course of tSN38 were modeled as a one-compartmental model with two input fluxes: from the initial amount of encapsulated SN-38, and from the in vivo conversion of un-encapsulated IRI released from nal-IRI. Compared to 120 mg/m² (salt) every 3 weeks, 80 mg/m² (salt) every 2 weeks resulted in similar average concentration and ⅓lower maximum concentration.

A total of 353 patients were used to develop the population PK model. Compared to 120 mg/m² (salt) every 3 weeks, 80 mg/m² (salt) every 2 weeks resulted in similar average concentration and a 1/3-lower Cmax.

Association between plasma PK and baseline covariates were evaluated for liver metastasis status, total bilirubin, AST, ALT, albumin, creatinine clearance (CrCI), pharmacogenetics (UGT1A1*28), age, sex, race, body surface area (BSA), coadministration with 5-FU/LV, and manufacturing site.

Race: Compared to Caucasians, Asians were observed to have lower tIRI and higher SN-38 UGT1A1*28: No significant association was observed. The prevalence of 7/7 homozygosity in Asians were low (1/85[1%]). Compared to non-7/7 Caucasians, 7/7 Caucasians had numerically higher (13%) uSN38 Cmax, but not statistically significant (these numbers were for a simulated dose of 80 mg/m² (salt) for both homozygous and non-homozygous patients; in NAPOLI-1, the dose in homozygous patients was lower [60 mg/m² in nal-IRI+5-FU/LV; 80 mg/m² (salt) in nal-IRI]). Separate analyses of patients with UGT1A1*28 6/6, 6/7, and 7/7 did not show a significant difference in the clearance of SN-38 (data not shown) for each of the UGT1A1*28 subgroups Bilirubin: Higher baseline bilirubin was associated with higher SN-38 concentration.

BSA: For tIRI, no association was observed with BSA; for SN-38, increased BSA was associated with lower Cmax. Simulation predicted that, compared to flat-dosing of 136 mg (the nominal dose for a subject with median BSA), a BSA-based dosing strategy would result in lower SN-38 PK variability (interquartile-range of 59% vs 74%)

In the nal-IRI+5-FU/LV arm of NAPOLI-1, longer OS and PFS were associated with higher Cavg of tIRI, tSN38, and uSN38, with the highest association observed for both tSN38 and uSN38. The relationship between OS and quartiles of uSN38 is provided in FIG. 5 and FIG. 8. Sensitivity analysis using adjustment for dose modification showed that the association between higher exposure and longer survival was maintained. Higher exposures were associated with higher probability of achieving objective response (OR) in the nal-IRI+5FU/LV arm (FIG. 6). The association was not observed in the nal-IRI monotherapy arm.

FIG. 5 is a Kaplan-Meier Plot of overall survival by quartiles of un-encapsulated SN-38 (uSN38) time above threshold in the nal-IRI+5-FU/LV arm of NAPOLI-1, and FIG. 8 is a Kaplan-Meyer Plot of Overall Survival (OS) by Quartiles of un-encapsulated SN-38 Average Concentration (Cavg) in the nal-IRI+5-FU/LV arm of Study.

FIG. 7 provides the association between Best Objective Response and average concentrations of total irinotecan and unencapsulated SN-38 in the nal-IRI+5-FU/LV arm of NAPOLI-1. PR=partial response, SD=stable disease, PD=progressive disease.

In the dataset of 353 patients, neutropenia is associated with uSN38 Cmax. The association with neutropenia was stronger for uSN38 Cmax than for tSN38 Cmax. Univariate analysis showed that uSN38 Cmax is associated with neutropenia, after adjusting for baseline absolute neutrophil count and co-administration with 5FU/LV (two known factors associated with neutropenia).

In the same dataset, diarrhea was associated with tIRI Cmax. The tIRI Cmax was observed at higher values for the nal-IRI monotherapy arm (120 mg/m² (salt) every 3 weeks) than for the nal-IRI+5FU/LV arm (80 mg/m² (salt) every 2 weeks) because of the difference in nal-IRI doses. The association was observed within the nal-IRI monotherapy arm, but not within the nal-IRI+5FU/LV arm; this is likely due to the higher tIRI Cmax values observed in the nal-IRI monotherapy arm than those in the nal-IRI+5FU/LV. Multivariate analysis showed that tIRI is associated with diarrhea in each of the Caucasian and Asian subgroups.

1. A mechanism-based population plasma PK analysis was developed for nal-IRI.

2. Un-encapsulated SN-38 Cmax is associated with neutropenia and is influenced by BSA, race, and bilirubin; total irinotecan Cmax is associated with diarrhea and is influenced by race.

3. In patients with metastatic pancreatic cancer previously treated with gemcitabine-based therapy (NAPOLI-1), higher total irinotecan and SN-38 plasma concentrations are associated with longer OS and PFS, and greater OR.

4. The population PK modeling shows that the nanoliposomal formulation of irinotecan (nal-IRI) confers a superior PK (lower uSN38 Cmax and longer half-life) than irinotecan HCl, while exerting significant anticancer benefits.

Converting a dose based on irinotecan hydrochloride trihydrate to a dose based on irinotecan free base is accomplished by multiplying the dose based on irinotecan hydrochloride trihydrate with the ratio of the molecular weight of irinotecan free base (586.68 g/mol) and the molecular weight of irinotecan hydrochloride trihydrate (677.19 g/mol). This ratio is 0.87 which can be used as a conversion factor. For example, an 80 mg/m² dose based on irinotecan hydrochloride trihydrate is equivalent to a 69.60 mg/m² dose based on irinotecan free base (80×0.87). In the clinic this is rounded to 70 mg/m² to minimize any potential dosing errors.

Doses of nal-IRI in these studies were calculated based on the equivalent dose of irinotecan hydrochloride trihydrate (salt); in this specification, unless specified otherwise, the doses are based on irinotecan as the free base. Accordingly, 70 mg/m² based on irinotecan as free base is equivalent to 80 mg/m² based on irinotecan as the hydrochloride trihydrate, and 100 mg/m² based on irinotecan as free base is equivalent to 120 mg/m² based on irinotecan as the hydrochloride trihydrate, in accordance with the table below.

| Salt | Free base |
|---|---|
| 180 | 150 |
| 120 | 100 |
| 100 | 90 |
| 80 | 70 |
| 60 | 50 |
| 50 | 45 |
| 40 | 35 |

### EXAMPLES

### Example 1

### In vitro efficacy of SN-38 in pancreatic cell lines

Background

Irinotecan (CPT-11) is a widely used chemotherapeutic, either used as in advanced late line disease in the palliative setting or early line in the curative setting. It is converted by carboxylesterases, primarily in liver and colon, to the active metabolite, SN-38.

Capello et al. (2015) recently reported on the sensitivity of pancreatic cell lines to irinotecan in vitro. The CFPAC-1 cell line was reported to have the lowest IC50 value with MiaPaCa-2<BxPC-3<AsPC1<Panc-1 ranked with increasing IC50 values.

The goal of this study was to test cytotoxic effects of SN-38 in a cell panel of pancreatic cancer. By using SN-38 instead of non-liposomal irinotecan, any differences in conversion ability of the different cell lines are avoided.

Result Summary

Exposure to SN-38 for 24 hours with a recovery period of 72 h led to significant cell killing in all pancreatic cell lines with IC50 ranging from 1 pM to 100 nM.

Panc-1, Capan-2 and Hs766t cell lines were the least sensitive to SN-38 with IC50 concentrations of 13.8 nM, 20.4nM and 63.1 nM, respectively.

Among cell lines for which in-house efficacy data had been obtained with MM-398 the CFPAC1 and MiaPaCa2 cell lines are the most sensitive with IC50 concentrations of 3.2 pM and 4.2 pM, respectively.

Materials & Methods

Culture/Treatment Condition

In vitro efficacy study was done using CellTiter-Glo® Luminescent Cell Viability Assay (Promega) with Corning Cat #3707 384well White Clear bottom plates. Cells were plated (1000 cells/well) in 384 well format and allowed to incubate @ 37 C for 24 hours. Monotherapy drugs were added at the 24 hr time point and then allowed to incubate @ 37 C for 24 hours. At the 48 hr time point the drugs in media were removed, washed with PBS, and fresh media was added. Cells were then allowed to incubate @ 37 C for 72 hours. At the 120 hr time point media was removed and CellTiter-Glo (CTG) reagent was added (1:1 ratio with PBS). Plate was read by luminometer (Envision Multilabel reader). More details of this assay can be found in Accelrys Notebook ELN as EXP-15-AC2111.

Cell Lines

Most pancreatic cell lines were obtained previously from ATCC (12) or RIKEN BRC (1). Select cell lines were obtained from collaborators at UCSF. Vials were thawed from our in-house Master Cell Bank collection (Table 21).

TABLE 21
Correlation of tissue type and source to each cell line.

| | Tissue | |
|---|---|---|
| Cell Line | Type | Source |
| Aspc1 | Pancreatic | ATCC/CRL-1682 |
| BxPC3 | Pancreatic | ATCC/CRL-1687 |
| Capan-2 | Pancreatic | ATCC/HTB-80 |
| CFPAC1 | Pancreatic | ATCC/CRL-1918 |
| Colo357 | Pancreatic | UCSF/I. Fidler |
| HPAFII | Pancreatic | ATCC/CRL-1997 |
| Hs766t | Pancreatic | ATCC/HTB-134 |
| KP4 | Pancreatic | Riken RCB1005 |
| L3.3 | Pancreatic | UCSF/I. Fidler |
| L3.6pL | Pancreatic | UCSF/I. Fidler |
| Miapaca2 | Pancreatic | ATCC/CRL-1420 |
| Panc1 | Pancreatic | ATCC/CRL-1469 |
| Panc6.03 | Pancreatic | ATCC/CRL-2550 |
| PL45 | Pancreatic | ATCC/CRL-2558 |
| SU8686 | Pancreatic | ATCC/CRL-1837 |
| SW1990 | Pancreatic | ATCC/CRL-2172 |

All cell lines had been adapted to and were maintained in RPMI with 10% fetal bovine serum and supplemented with penicillin/streptomycin (Invitrogen).

Data Analysis

Data was analyzed using an in-house algorithm developed using Matlab (Mathworks, Natick MA). In summary, average CTG mean luminescent values were computed for 4 replicate wells. Outlier detection was performed by computing the coefficient of variation (CV>20%) and outliers were removed from the average. CTG values were normalized based on a control non-treated well. Drug concentration in microMolar (uM) was log transformed prior to fitting to a 4 parameter logistic curve.

$$y = b + \frac{( a - b )}{( 1 + {10}^{( IC 50 - C ) * slope} )}$$

Data quality control was performed to ensure that the concentration range is optimal according to these rules: (1) if the lowest concentration kills more than 70% of the cells the concentration range is deemed too potent (2) if the highest concentration kills less than 30% of the cells, the concentration range is deemed low or the cell line is too resistant. Additionally, goodness of the fit was evaluated using R² and R²<0.9 is flagged as a bad fit. Statistical analysis was performed using JMP (SAS Institute Inc., NC).

Results

Data Analysis Summary—Curve Fits

Pancreatic cell lines were cultured to sub confluent state in 384 well plates prior to incubation with varying doses of SN-38 for 24 hours. Additions 72 hours of culture were performed prior to CTG assessment. Every well was run in 4 replicates, and the entire experiment was run in four different plates. The compared results from the four separate experiments show the reproducibility of the cytotoxic effects of SN-38. SN-38 induced a decrease in cell viability of around 90% for most cell lines, the IC50 was variable and spanned 5 orders of magnitude.

Data Analysis Summary—Summary of IC50 and Tumor Cell Kill

TABLE 22
IC50 and Tumor Cell Kill in pancreatic cell lines

| | | | | | Tumor Cell | |
|---|---|---|---|---|---|---|
| | IC50 Log(μM) | | | IC50(nM) | Kill (%) | |
| Cell Line | n | mean | sem | mean | mean | sem |
| Aspc1 | 3 | −2.40 | 0.30 | 3.9811 | 91.61 | 4.57 |
| BxPC3 | 2 | −4.77 | 0.33 | 0.0170 | 77.37 | 1.00 |
| Capan-2 | 3 | −1.69 | 0.07 | 20.4174 | 94.61 | 2.75 |
| CFPAC1 | 2 | −5.50 | 0.06 | 0.0032 | 84.74 | 4.06 |
| Colo357 | 2 | −4.21 | 0.10 | 0.0617 | 94.47 | 0.69 |
| HPAFII | 3 | −2.69 | 0.29 | 2.0417 | 93.24 | 1.92 |
| Hs766t | 2 | −1.20 | 0.07 | 63.0957 | 86.41 | 2.89 |
| KP4 | 2 | −5.10 | 0.09 | 0.0079 | 86.62 | 3.05 |
| L3.3 | 2 | −5.69 | 0.27 | 0.0020 | 93.01 | 1.75 |
| L3.6pL | 2 | −5.55 | 0.10 | 0.0028 | 95.18 | 1.72 |
| Miapaca2 | 3 | −5.38 | 0.50 | 0.0042 | 91.23 | 3.26 |
| Panc1 | 4 | −1.86 | 0.31 | 13.8038 | 89.54 | 3.95 |
| Panc6.03 | 4 | −2.94 | 0.24 | 1.1482 | 87.68 | 5.58 |
| PL45 | 2 | −5.45 | 0.14 | 0.0035 | 92.16 | 0.34 |
| SU8686 | 2 | −3.16 | 0.05 | 0.6918 | 95.82 | 4.18 |
| SW1990 | 4 | −4.06 | 0.42 | 0.0871 | 91.92 | 4.67 |

Tumor cell kill rates averaged 90% across all cell lines. Lowest kill rates were observed for BxPC3 and CFPAC-1 cell lines. Highest kill rates were observed for SU8686 and L3.6pL cell lines.

Lowest IC50 concentrations were observed for L3.3, L3.6pL and CFPAC-1 cell lines. Highest IC50 concentrations were observed for Hs766t, Capan-2 and Panc-1 cell lines.

### Example 2

### Plasma Pharmacokinetics of Liposomal Irinotecan (nal-IRI) in Pediatric Oncology Patients with Recurrent or Refractory Solid Tumors

Background

Children with relapsed or refractory solid tumors have a poor prognosis. Irinotecan is active in some pediatric solid tumors and synergizes with alkylating agents. nal-IRI encapsulates irinotecan into long-circulating, liposome-based nanoparticles. In adults, nal-IRI demonstrated extended plasma exposure compared with non-liposomal irinotecan. In pediatric solid tumor models, nal-IRI had robust preclinical activity and synergized with cyclophosphamide, and therefore merits testing in children with relapsed and refractory solid tumors. Also described herein is a phase 1 dose-escalation study of nal-IRI in combination with cyclophosphamide and preliminary pharmacokinetic and safety results in pediatric patients.

Methods and Materials

Cyclophosphamide was administered on days 1-5 of each cycle (250 mg/m²/d intravenously [IV]) with a single 90-min IV infusion of nal-IRI on day 3 of a Q3-week schedule, escalating from 60 mg/m² to 210 mg/m² (expressed as irinotecan HCL trihydrate salt), in a standard 3+3 dose-escalation design to determine the maximum tolerated dose. To date, the nal-IRI dose has been escalated from 60 mg/m² to 150 mg/m². Samples for pharmacokinetic analysis were collected during the first cycle of chemotherapy before infusion and at 4 h, 24 h, 48 h, 120 h, and 168 h post-infusion. Plasma pharmacokinetics of total irinotecan and SN-38 were quantified using mixed effect modeling, and were compared with adult values from a population pharmacokinetic analysis of 6 clinical studies of nal-IRI.

### Results

To date, 10 males and 6 females with a median age of 12.8 years (range: 5-19) have been enrolled: 10 with Ewing sarcoma, 2 with neuroblastoma, 3 with osteosarcoma, and 1 with rhabdomyosarcoma. The estimated total irinotecan volume of distribution (V_{d}) was 1.9 L, clearance (CL) was 10.3 L/week, and half-life (t_{1/2}) was 21.2 h, which were 42% (V_{d} and CL) of adult values and comparable to adult values (t_{1/2}). The corresponding Cₘₐₓ was 72% higher than that observed in adults. SN-38 clearance was 11.4 L/week (comparable to adults), t_{1/2} was 19.3 h (48% of adult values), and Cₘₐₓ was 68% of adult values. Thrombocytopenia leading to treatment delay was a dose-limiting toxicity (n=1) at 150 mg/m²; other systemic toxicity attributed to chemotherapy within the ^{1st} cycle was nausea/vomiting (n=1).

What is claimed is:

1. A method of treating cancer in a human patient, the method comprising administering to the human patient in need thereof irinotecan liposome in a dose and dose interval that are both therapeutically tolerable and therapeutically effective, wherein
a. the therapeutically tolerable dose and dose interval is selected based on the maximum SN38 plasma concentration and the maximum total irinotecan concentration in the plasma of the patient, and
b. the therapeutically effective dose and dose interval is selected based on the time of SN38 plasma concentration above a cancer indication-specific threshold concentration, and the average SN38 plasma concentration of the patient.

2. The method of claim 1, wherein the irinotecan liposome is MM-398.

3. The method of claim 1 or 2, wherein the therapeutically tolerable dose and dose interval are selected to provide a minimal predicted incidence of neutropenia and diarrhea at a given therapeutically effective dose and dose interval.

4. The method of any one of claims 1-3, wherein the cancer comprises a solid tumor in the human patient.

5. The method of any one of claims 1-4, wherein the cancer is pancreatic cancer.

6. A method of treating cancer in a human patient, the method comprising administering to the human patient in need thereof irinotecan liposome in a first dose that is both therapeutically tolerable and therapeutically effective, followed by administering a second dose of the irinotecan liposome at a first dose interval after the first dose, wherein the second dose and first dose interval are selected based on:
a. the maximum unencapsulated SN38 plasma concentration and the maximum total irinotecan concentration in the plasma of the patient, and
b. the therapeutically effective dose and dose interval is selected based on the time of SN38 plasma concentration above a cancer indication-specific threshold concentration, and the average SN38 plasma concentration of the patient.

7. The method of claim 6, further comprising measuring the total irinotecan and the SN-38 in the plasma of the human patient after the first dose and before the second dose.

8. The method of claim 7, further comprising administering a third dose following a second dose interval after the second dose, wherein the second dose interval is determined using the measurement of the total irinotecan and the SN-38 in the plasma of the human patient after the first dose and before the second dose.

9. The method of any one of claims 1-8, wherein
a. the maximum SN38 plasma concentration is from 0.88 to 5.98 ng/mL,
b. the maximum total irinotecan concentration in the plasma of the patient is from 18.9 mg/L to 53.1 mg/L,
c. the time of SN38 plasma concentration above the threshold concentration of 0.03 ng/mL in the first 6 weeks is from 1.94 to 6.00 weeks, and
d. the average SN38 plasma concentration of the patient is from 0.20 to 1.66 ng/mL.

10. The method of any one of claims 1-9, wherein the human patient is a pediatric patient.

11. The method of claim 10, wherein the pediatric patient is from 5-19 years old.

12. The method of claim 11, wherein the pediatric patient is from 10-15 years old.

13. The method of any one of claims 10-12, wherein one or more doses of liposome irinotecan is administered in combination with cyclophosphamide.
